(19) United States
(12) Patent Application Publication
(10) Pub. No.: US 2023/0304066 A1
(43) Pub. Date: Sep. 28, 2023

(54) MICROFLUIDIC BASED ASSAY FOR UNBOUND BILIRUBIN

(71) Applicant: Baebies, Inc., Durham, NC (US)

(72) Inventors: Michael A. BASMAJIAN, Durham, NC (US); Jennifer ELDERBROOM, Durham, NC (US); Maithri KRISHNAMURTHY, Durham, NC (US); Erik BURDE, Durham, NC (US); Michael BOSO, Durham, NC (US); Daniel Hull, Durham, NC (US); Rainer NG, Durham, NC (US); Vijay SRINIVASAN, Durham, NC (US); Ramakrishna SISTA, Durham, NC (US); Vamsee PAMULA, Durham, NC (US)

(73) Assignee: Baebies, Inc., Durham, NC (US)

(21) Appl. No.: 18/020,883

(22) PCT Filed: Sep. 7, 2021
(86) PCT No.: PCT/US2021/049203

§ 371 Date: Feb. 10, 2023

Related U.S. Application Data
(60) Provisional application No. 63/123,138, filed on Dec. 9, 2020.
(60) Provisional application No. 63/074,580, filed on Sep. 4, 2020.

(51) Int. Cl. C12Q 1/28; B01L 3/00

(52) U.S. Cl. CPC C12Q 1/28; B01L 3/502715; B01L 2200/16; B01L 2400/0427; B01L 2200/04; G01N 2333/908; G01N 2800/085

(57) ABSTRACT

A method for assaying analytes in a blood sample by loading a blood sample onto a microfluidic device; combining the blood sample with a buffer reagent comprising a surfactant to provide a diluted blood sample and conduct an assay. The surfactant is selected to permit the use of electrowetting to conduct droplet operations using the blood sample and to permit the use of a fluorescence-based droplet operation. The assay may be an unbound bilirubin assay.

### 1. RELATED APPLICATIONS

This application claims priority to and the benefit of U.S. Provisional Patent App. No. 63/074,580 filed on Sep. 4, 2020 and U.S. Provisional Patent App. No. 63/123,138 filed on Dec. 9, 2020; the disclosures of which are hereby incorporated herein by reference.

### 2. FIELD OF THE INVENTION

The subject matter relates to apparatuses and methods for screening newborns for high bilirubin levels in the blood (hyperbilirubinemia) and more particularly to screening newborns for high levels of unbound unconjugated (indirect) bilirubin in the blood utilizing a microfluidics platform.

### 3. BACKGROUND OF THE INVENTION

Bilirubin screening is a common practice to screen newborns for high bilirubin levels in the blood (hyperbilirubinemia). Bilirubin can be present in the blood in two forms, bound and unbound. Bound bilirubin is unconjugated (or indirect) bilirubin that is bound to albumin. Unbound bilirubin includes unconjugated (indirect) bilirubin that is not bound to albumin and conjugated (direct) bilirubin. For newborns, it is high levels of unbound unconjugated (indirect) bilirubin that can be problematic because the molecule is relatively small and can cross the blood-brain barrier causing a condition known as kernicterus.

Various assays have been developed to measure unbound bilirubin in a blood sample. For example, an oxidative decomposition reaction can be used to oxidize unbound bilirubin and the rate of absorbance loss is then used to determine how much unbound bilirubin is in a sample (Jacobsen, J. and R. P. Wennberg, Clin Chem. (1974) 20(7):783, and Nakamura, H., and Y. Lee, Clin Chim Acta. (1977) 79(2): 411-417, which are incorporated herein by reference in their entirety). However, the oxidative decomposition methods are not specific for unconjugated bilirubin.

More recently, a fluorescence-based method for detecting unconjugated bilirubin using a protein, UnaG, that is specific for unconjugated bilirubin has been described (Kumagai, A., et. al., Cell (2013) 153:1602-1611, which is incorporated herein by reference in its entirety). A method of screening for unbound bilirubin using a combination of oxidative decomposition and UnaG binding has been described (International App. No. PCT/JP2016/060327, entitled “Measurement method for unbound bilirubin in blood sample”, which is incorporated herein by reference in its entirety). However, the current technologies for screening for unbound (unconjugated) bilirubin are not readily provided in a point-of-birth testing system.

### 4. SUMMARY OF THE INVENTION

The invention provides a method for assaying unbound bilirubin in a blood sample. The method may include dispensing one or more sample droplets from a blood sample or diluted blood sample in a droplet operations gap of the microfluidic device. The method may include Initiating a biochemical assay on each of the oner or more sample droplets to detect unbound bilirubin in the diluted blood sample droplet. The sample droplets may, for example, each have a volume less than about 5 mL. The microfluidic device may be an electrowetting cartridge. The loading, combining, dispensing, and/or initiating may be performed using electrowetting-mediated droplet operations. The blood sample may, for example, be whole blood, plasma or serum, or a processed or diluted version of any of the foregoing.

The invention provides a method for assaying analytes in a blood sample. The method may, for example, include loading a blood sample having one or more analytes to be assayed onto a microfluidic device. The method may, for example, include combining the blood sample with a buffer reagent having a surfactant to provide a diluted blood sample, wherein the surfactant may, for example, be selected to permit electrowetting to conduct droplet operations using the blood sample. The method may, for example, include dispensing one or more sample droplets from the diluted blood sample in a droplet operations gap of the microfluidic device, the gap having an oil filler fluid, thereby providing a diluted blood sample droplet. The method may, for example, include transporting a diluted blood sample droplet to an assay reaction zone. The method may, for example, include initiating a biochemical assay. The buffer reagent may, for example, include a glucose reagent.

The blood sample may, for example, include a whole blood sample. The method may, for example, include processing the diluted blood sample to provide a processed blood sample having one or more analytes to be assayed. The processing may, for example, include lysing the diluted blood sample. The processed blood sample may, for example, include a blood component. The blood component may, for example, be plasma.

The surfactant may, for example, be selected to provide permit electrowetting a whole blood sample droplet without causing significant lysis of the whole blood sample. The surfactant may, for example, be selected to minimize or eliminate a fluorescence signal. The surfactant may, for example, include a non-ionic surfactant. The non-ionic surfactant may, for example, include Tween® 80. The non-ionic surfactant may, for example, include Facade®-TEM. The surfactant may, for example, include a zwitterionic surfactant. The zwitterionic surfactant may, for example, include 11:0 Lyso PC.

The biochemical assay may, for example, be an enzymatic, fluorescence-based assay for measuring unbound bilirubin in a plasma droplet.

The method provides a method of measuring unbound bilirubin in a plasma droplet. The method may, for example, include providing a plasma droplet having a glucose buffer and a surfactant compatible with performing a fluorescence-based unbound bilirubin assay using electrowetting-mediated droplet operations to perform assay steps. The method may, for example, include splitting the plasma droplet into at least three sample droplets and initiating the unbound bilirubin assay. A first sample droplet may, for example, be combined with a buffer droplet to provide a control reaction droplet. A second sample droplet may, for example, be combined with an enzyme reagent droplet to provide a short reaction droplet. A third sample droplet may, for example, be combined with a second enzyme reagent droplet to provide a long reaction droplet. The method may, for example, include combining the control reaction droplet and the short reaction droplet with a stop reaction droplet at a time t1 to provide a control reacted droplet and a short-reacted droplet, wherein any unbound bilirubin in the short-reacted droplet may, for example, be oxidized, thereby providing a t1 decomposition product droplet. The method may, for example, include combining the long reaction droplet with a stop reaction droplet at a time t2 to provide a long-reacted droplet, wherein any remaining unbound bilirubin in the long-reacted droplet may, for example, be oxidized, thereby providing a t2 decomposition product droplet. The method may, for example, include diluting the control reacted droplet, t1 decomposition product droplet and t2 decomposition product droplet with a buffer reagent to provide a diluted control reacted droplet, a diluted t1 decomposition product droplet, and a diluted t2 decomposition product droplet for combining with a detection reagent. The method may, for example, include combining the diluted control reacted, t1 decomposition product, and t2 decomposition product droplets with a detection reagent to provide a control/detection reagent droplet, a short reacted/detection reagent droplet and a long reacted/detection reagent droplet. The method may, for example, include detecting a reaction product in the control/detection reagent droplet, short reacted/detection reagent droplet, and long reacted/detection reagent droplet to determine the amount of unbound bilirubin in the plasma droplet.

The enzyme reagent droplet may, for example, include glucose oxidase (GOD) and peroxidase (POD). The stop reaction droplet may, for example, include ascorbic acid. Time t1 may, for example, be about 48 seconds. The time t2 may, for example, be about 120 seconds.

Combining each diluted reaction droplet with a detection reagent may, for example, include transporting a diluted reaction droplet to a certain droplet operations electrode having a dried detection reagent and reconstituting the dried detection reagent. The dried detection reagent for detecting unbound bilirubin may, for example, be UnaG. Detecting a reaction product may, for example, include measuring a UnaG fluorescence signal.

Determining the amount of unbound bilirubin in the plasma droplet may, for example, include determining the difference in the UnaG fluorescence signal between the control/detection reagent droplet, and short and long reacted/detection reagent droplets.

The invention provides a system having a computer processor and an electrowetting cartridge wherein the processor may, for example, be programmed to execute the method of any one of methods of the invention on the cartridge. The cartridge may, for example, be an electrowetting-mediated droplet operations device. The invention provides a kit having an electrowetting cartridge and reagents sufficient to execute any of the methods of the invention on an electrowetting-mediated droplet operations device.

### 5. BRIEF DESCRIPTION OF DRAWINGS

FIG. 1 is a cross-sectional view illustrating an example of a portion of a microfluidics device for performing a biochemical assay in droplets.

FIG. 2 is a schematic diagram illustrating an example of an arrangement of droplet operations electrodes configured for conducting a GOD-POD-UnaG assay for unbound bilirubin on a microfluidic device.

FIG. 3 is a flow diagram illustrating an example of a method for measuring unbound bilirubin in a blood sample using a GOD-POD-UnaG assay on a microfluidic device.

FIG. 4 are plots showing the % bias of RFUs at t=3 minutes for the 96 detergents tested.

FIG. 5 is a table showing the microtiter plate layout of the 96 detergents in the Detergent Screen kit used to screen for interference in the UnaG-bilirubin fluorescence assay.

FIG. 6A and FIG. 6B are plots showing a spectral scan for hemoglobin in the n=35 surfactant samples.

FIG. 7 is a screenshot showing the data sheet for the priming/dispensing assessment of some of the n=35 down selected surfactants.

FIG. 8 is a screenshot showing the data sheet for the electrowetting assessment of the second down selected set of n=8 surfactants using modified surfactant concentrations.

FIG. 9A is a table showing the percent bias from control for the 7 surfactants and 12 plasma samples used to screen for fluorescence interference.

FIG. 9B is a plot showing percent bias from the no surfactant control at t=3 minutes for the 7 surfactants tested.

FIG. 10A is a table showing the percent bias from control at t=3 minutes for the 3 surfactants and 10 plasma samples used to screen for fluorescence interference.

FIG. 1013 is a table showing the percent bias from control at t=10 minutes for the 3 surfactants and 10 plasma samples used to screen for fluorescence interference.

FIG. 11 is a table showing RFUs over time for the on-bench fluorescence interference assay used to assess variability.

FIG. 12 is a plot showing an example of an UnaG-bilirubin binding assay performed using the modified fluorescence interference assay.

FIG. 13A is a plot showing the RFU over time in reactions using the 5.5 mg/dL TBIL plasma sample and the surfactants Tween® 20 or Tween® 80.

FIG. 132B is a plot showing the RFU over time in reactions using the 5.5 mg/dL TBIL plasma sample and the surfactant Facade®-TEM.

FIG. 14A is a table showing the percent bias from control at t=3 minutes for Tween® 80, Facade®-TEM, and Tween® 20 in the UnaG fluorescence assay.

FIG. 14B is a table showing the percent bias from control at t=10 minutes for Tween® 80, Facade®-TEM, and Tween® 20 in the UnaG fluorescence assay.

FIG. 15A is a plot showing the RFU over time for the n=2 reactions using the 13.1 mg/dL TBIL plasma sample and no surfactant control.

FIG. 15B is a plot showing the RFU over time for the n=2 reactions using the 13.1 mg/dL TBIL plasma sample and 0.1% Tween® 20.

FIG. 15C is a plot showing the RFU over time for the n=2 reactions using the 13.1 mg/dL TBIL plasma sample and 0.1% Tween® 80.

FIG. 16 is a table showing the assay values for G6PD, albumin (ALB), and TBIL for the Tween® 20, Tween® 80, and Facade®-TEM runs.

FIG. 17 is a table showing the percent bias from control for Tween® 20, Tween® 80, and Facade®-TEM in the GOD-POD-UnaG fluorescence assay.

FIG. 18A is a plot showing a comparison of the RFU (control-reacted sample) values relative to the Arrow UB Analyzer assigned value for Tween® 20.

FIG. 18B is a plot showing a comparison of the RFU (control-reacted sample) values relative to the Arrow UB Analyzer assigned value for Tween® 80.

FIG. 18C is a plot showing a comparison of the RFU (control-reacted sample) values relative to the Arrow UB Analyzer assigned value for Facade®-TEM

FIG. 19 is an enlargement of the plot of FIG. 18B showing the comparison of the lower three data points for the RFU (control-reacted sample) values relative to the Arrow UB Analyzer assigned value for Tween® 80.

FIG. 20 is a table showing the RFU values for the GOD-POD-UnaG assays performed on-cartridge and the “control-reacted” RFU values for the GOD-POD-UnaG assay performed on-bench.

FIG. 21A is a plot showing the RFU (control-reacted sample) values vs DLS TBIL obtained on-cartridge using Tween® 20 and Tween® 80.

FIG. 21B is a plot showing the RFU (control-reacted sample) values obtained on-cartridge using Tween® 20 and Tween® 80 relative to the RFU obtained in the on-bench assay.

### 6. DETAILED DESCRIPTION OF THE INVENTION

### 6.1. Definitions

As used here the following terms have the meanings indicated:

“Activate,” with reference to one or more electrodes, means affecting a change in the electrical state of the one or more electrodes which, in the presence of a droplet, results in a droplet operation. Activation of an electrode can be accomplished using alternating or direct current. Any suitable voltage may be used. For example, an electrode may be activated using a voltage which is greater than about 150 V, or greater than about 200 V, or greater than about 250 V, or from about 275 V to about 1000 V, or about 300 V. Where alternating current is used, any suitable frequency may be employed. For example, an electrode may be activated using alternating current having a frequency from about 1 Hz to about 10 MHz, or from about 10 Hz to about 60 Hz, or from about 20 Hz to about 40 Hz, or about 30 Hz.

“Droplet Actuator” means a device for manipulating droplets. For examples of droplet actuators, see Pamula et al., U.S. Pat. No. 6,911,132, entitled “Apparatus for Manipulating Droplets by Electrowetting-Based Techniques,” issued on Jun. 28, 2005; Pamula et al., U.S. patent application Ser. No. 11/343,284, entitled “Apparatuses and Methods for Manipulating Droplets on a Printed Circuit Board,” filed on Jan. 30, 2006; Pollack et al., International Patent Application No. PCT/US2006/047486, entitled “Droplet-Based Biochemistry,” filed on Dec. 11, 2006; Shenderov, U.S. Pat. No. 6,773,566, entitled “Electrostatic Actuators for Microfluidics and Methods for Using Same,” issued on Aug. 10, 2004 and U.S. Pat. No. 6,565,727, entitled “Actuators for Microfluidics Without Moving Parts,” issued on Jan. 24, 2000; Kim and/or Shah et al., U.S. patent application Ser. No. 10/343,261, entitled “Electrowetting-driven Micropumping,” filed on Jan. 27, 2003, Ser. No. 11/275,668, entitled “Method and Apparatus for Promoting the Complete Transfer of Liquid Drops from a Nozzle,” filed on Jan. 23, 2006, Ser. No. 11/460,188, entitled “Small Object Moving on Printed Circuit Board,” filed on Jan. 23, 2006, Ser. No. 12/465,935, entitled “Method for Using Magnetic Particles in Droplet Microfluidics,” filed on May 14, 2009, and Ser. No. 12/513,157, entitled “Method and Apparatus for Real-time Feedback Control of Electrical Manipulation of Droplets on Chip,” filed on Apr. 30, 2009; Velev, U.S. Pat. No. 7,547,380, entitled “Droplet Transportation Devices and Methods Having a Fluid Surface,” issued on Jun. 16, 2009; Sterling et al., U.S. Pat. No. 7,163,612, entitled “Method, Apparatus and Article for Microfluidic Control via Electrowetting, for Chemical, Biochemical and Biological Assays and the Like,” issued on Jan. 16, 2007; Becker and Gascoyne et al., U.S. Pat. No. 7,641,779, entitled “Method and Apparatus for Programmable fluidic Processing,” issued on Jan. 5, 2010, and U.S. Pat. No. 6,977,033, entitled “Method and Apparatus for Programmable Fluidic Processing,” issued on Dec. 20, 2005; Decre et al., U.S. Pat. No. 7,328,979, entitled “System for Manipulation of a Body of Fluid,” issued on Feb. 12, 2008; Yamakawa et al., U.S. Patent Pub. No. 20060039823, entitled “Chemical Analysis Apparatus,” published on Feb. 23, 2006; Wu, International Patent Pub. No. WO/2009/003184, entitled “Digital Microfluidics Based Apparatus for Heat-exchanging Chemical Processes,” published on Dec. 31, 2008; Fouillet et al., U.S. Patent Pub. No. 20090192044, entitled “Electrode Addressing Method,” published on Jul. 30, 2009; Fouillet et al., U.S. Pat. No. 7,052,244, entitled “Device for Displacement of Small Liquid Volumes Along a Micro-catenary Line by Electrostatic Forces,” issued on May 30, 2006; Marchand et al., U.S. Patent Pub. No. 20080124252, entitled “Droplet Microreactor,” published on May 29, 2008; Adachi et al., U.S. Patent Pub. No. 20090321262, entitled “Liquid Transfer Device,” published on Dec. 31, 2009; Roux et al., U.S. Patent Pub. No. 20050179746, entitled “Device for Controlling the Displacement of a Drop Between two or Several Solid Substrates,” published on Aug. 18, 2005; Dhindsa et al., “Virtual Electrowetting Channels: Electronic Liquid Transport with Continuous Channel Functionality,” Lab Chip, 10:832-836 (2010); the entire disclosures of which are incorporated herein by reference, along with their priority documents.

Certain droplet actuators will include one or more substrates arranged with a droplet operations gap therebetween and electrodes associated with (e.g., layered on, attached to, and/or embedded in) the one or more substrates and arranged to conduct one or more droplet operations. For example, certain droplet actuators will include a base (or bottom) substrate, droplet operations electrodes associated with the substrate, one or more dielectric layers atop the substrate and/or electrodes, and optionally one or more hydrophobic layers atop the substrate, dielectric layers and/or the electrodes forming a droplet operations surface.

A top substrate may also be provided, which is separated from the droplet operations surface by a gap, commonly referred to as a droplet operations gap. Various electrode arrangements on the top and/or bottom substrates are discussed in the above-referenced patents and applications and certain novel electrode arrangements are discussed in the description of the invention. During droplet operations it is preferred that droplets remain in continuous contact or frequent contact with a ground or reference electrode. A ground or reference electrode may be associated with the top substrate facing the gap, the bottom substrate facing the gap, in the gap. Where electrodes are provided on both substrates, electrical contacts for coupling the electrodes to a droplet actuator instrument for controlling or monitoring the electrodes may be associated with one or both plates. In some cases, electrodes on one substrate are electrically coupled to the other substrate so that only one substrate is in contact with the droplet actuator.

In one embodiment, a conductive material (e.g., an epoxy, such as MASTER BOND™ Polymer System EP79, available from Master Bond, Inc., Hackensack, N.J.) provides the electrical connection between electrodes on one substrate and electrical paths on the other substrates, e.g., a ground electrode on a top substrate may be coupled to an electrical path on a bottom substrate by such a conductive material.

Where multiple substrates are used, a spacer may be provided between the substrates to determine the height of the gap therebetween and define dispensing reservoirs. The spacer height may, for example, be from about 5 μm to about 600 μm, or about 100 μm to about 400 μm, or about 200 μm to about 350 μm, or about 250 μm to about 300 μm, or about 275 μm. The spacer may, for example, be formed of a layer of projections from the top or bottom substrates, and/or a material inserted between the top and bottom substrates.

One or more openings may be provided in the one or more substrates for forming a fluid path through which liquid may be delivered into the droplet operations gap. The one or more openings may in some cases be aligned for interaction with one or more electrodes, e.g., aligned such that liquid flowed through the opening will come into sufficient proximity with one or more droplet operations electrodes to permit a droplet operation to be affected by the droplet operations electrodes using the liquid. The base (or bottom) and top substrates may in some cases be formed as one integral component. One or more reference electrodes may be provided on the base (or bottom) and/or top substrates and/or in the gap. Examples of reference electrode arrangements are provided in the above referenced patents and patent applications.

In various embodiments, the manipulation of droplets by a droplet actuator may be electrode mediated, e.g., electrowetting mediated or dielectrophoresis mediated or Coulombic force mediated.

Examples of other techniques for controlling droplet operations that may be used in the droplet actuators of the invention include using devices that induce hydrodynamic fluidic pressure, such as those that operate on the basis of mechanical principles (e.g. external syringe pumps, pneumatic membrane pumps, vibrating membrane pumps, vacuum devices, centrifugal forces, piezoelectric/ultrasonic pumps and acoustic forces); electrical or magnetic principles (e.g. electroosmotic flow, electrokinetic pumps, ferrofluidic plugs, electrohydrodynamic pumps, attraction or repulsion using magnetic forces and magnetohydrodynamic pumps); thermodynamic principles (e.g. gas bubble generation/phase-change-induced volume expansion); other kinds of surface-wetting principles (e.g. electrowetting and optoelectrowetting, as well as chemically, thermally, structurally, and radioactively induced surface-tension gradients); gravity; surface tension (e.g., capillary action); electrostatic forces (e.g., electroosmotic flow); centrifugal flow (substrate disposed on a compact disc and rotated); magnetic forces (e.g., oscillating ions causes flow); magnetohydrodynamic forces; and vacuum or pressure differential.

In certain embodiments, combinations of two or more of the foregoing techniques may be employed to conduct a droplet operation in a droplet actuator of the invention. Similarly, one or more of the foregoing may be used to deliver liquid into a droplet operations gap, e.g., from a reservoir in another device or from an external reservoir of the droplet actuator (e.g., a reservoir associated with a droplet actuator substrate and a flow path from the reservoir into the droplet operations gap).

Droplet operations surfaces of certain droplet actuators of the invention may be made from hydrophobic materials or may be coated or treated to make them hydrophobic. For example, in some cases some portion or all of the droplet operations surfaces may be derivatized with low surface-energy materials or chemistries, e.g., by deposition or using in situ synthesis using compounds such as poly- or per-fluorinated compounds in solution or polymerizable monomers. Examples include TEFLON® AF (available from DuPont, Wilmington, Del.), members of the cytop family of materials, coatings in the FLUOROPEL® family of hydrophobic and superhydrophobic coatings (available from Cytonix Corporation, Beltsville, Md.), silane coatings, fluorosilane coatings, hydrophobic phosphonate derivatives (e.g., those sold by Aculon, Inc), and NOVEC™ electronic coatings (available from 3M Company, St. Paul, Minn.), other fluorinated monomers for plasma-enhanced chemical vapor deposition (PECVD), and organosiloxane (e.g., SiOC) for PECVD. In some cases, the droplet operations surface may include a hydrophobic coating having a thickness ranging from about 10 nm to about 1,000 nm.

Moreover, in some embodiments, the top substrate of the droplet actuator includes an electrically conducting organic polymer, which is then coated with a hydrophobic coating or otherwise treated to make the droplet operations surface hydrophobic. For example, the electrically conducting organic polymer that is deposited onto a plastic substrate may be poly(3,4-ethylenedioxythiophene) poly(styrenesulfonate) (PEDOT:PSS). Other examples of electrically conducting organic polymers and alternative conductive layers are described in Pollack et al., International Patent Application No. PCT/US2010/040705, entitled “Droplet Actuator Devices and Methods,” the entire disclosure of which is incorporated herein by reference.

One or both substrates may be fabricated using a printed circuit board (PCB), glass, indium tin oxide (ITO)-coated glass, and/or semiconductor materials as the substrate. When the substrate is ITO-coated glass, the ITO coating is preferably a thickness in the range of about 20 to about 200 nm, preferably about 50 to about 150 nm, or about 75 to about 125 nm, or about 100 nm.

In some cases, the top and/or bottom substrate includes a PCB substrate that is coated with a dielectric, such as a polyimide dielectric, which may in some cases also be coated or otherwise treated to make the droplet operations surface hydrophobic. When the substrate includes a PCB, the following materials are examples of suitable materials: MITSUI™ BN-300 (available from MITSUI Chemicals America, Inc., San Jose Calif.); ARLON™ 11N (available from Arlon, Inc, Santa Ana, Calif.).; NELCO® N4000-6 and N5000-30/32 (available from Park Electrochemical Corp., Melville, N.Y.); ISOLA™ FR406 (available from Isola Group, Chandler, Ariz.), especially 6620; fluoropolymer family (suitable for fluorescence detection since it has low background fluorescence); polyimide family; polyester; polyethylene naphthalate; polycarbonate; polyetheretherketone; liquid crystal polymer; cyclo-olefin copolymer (COC); cyclo-olefin polymer (COP); aramid; THERMOUNT® non-woven aramid reinforcement (available from DuPont, Wilmington, Del.); NOMEX® brand fiber (available from DuPont, Wilmington, Del.); and paper.

Various materials are also suitable for use as the dielectric component of the substrate. Examples include: vapor deposited dielectric, such as PARYLENE™ C (especially on glass), PARYLENE™ N, and PARYLENE™ HT (for high temperature, −300° C.) (available from Parylene Coating Services, Inc., Katy, Tex.); TEFLON® AF coatings; cytop; soldermasks, such as liquid photoimageable solder masks (e.g., on PCB) like TAIYO™ PSR4000 series, TAIYO™ PSR and AUS series (available from Taiyo America, Inc. Carson City, Nev.) (good thermal characteristics for applications involving thermal control), and PROBIMER™ 8165 (good thermal characteristics for applications involving thermal control (available from Huntsman Advanced Materials Americas Inc., Los Angeles, Calif.); dry film solder mask, such as those in the VACREL® dry film solder mask line (available from DuPont, Wilmington, Del.); film dielectrics, such as polyimide film (e.g., KAPTON® polyimide film, available from DuPont, Wilmington, Del.), polyethylene, and fluoropolymers (e.g., FEP), polytetrafluoroethylene; polyester; polyethylene naphthalate; cyclo-olefin copolymer (COC); cyclo-olefin polymer (COP); any other PCB substrate material listed above; black matrix resin; and polypropylene.

Droplet transport voltage and frequency may be selected for performance with reagents used in specific assay protocols. Design parameters may be varied, e.g., number and placement of on-actuator reservoirs, number of independent electrode connections, size (volume) of different reservoirs, placement of magnets/bead washing zones, electrode size, inter-electrode pitch, and gap height (between top and bottom substrates) may be varied for use with specific reagents, protocols, droplet volumes, etc. In some cases, a substrate of the invention may be derivatized with low surface-energy materials or chemistries, e.g., using deposition or in situ synthesis using poly- or per-fluorinated compounds in solution or polymerizable monomers. Examples include TEFLON® AF coatings and FLUOROPEL® coatings for dip or spray coating, other fluorinated monomers for plasma-enhanced chemical vapor deposition (PECVD), and organosiloxane (e.g., SiOC) for PECVD.

Additionally, in some cases, some portion or all of the droplet operations surface may be coated with a substance for reducing background noise, such as background fluorescence from a PCB substrate. For example, the noise-reducing coating may include a black matrix resin, such as the black matrix resins available from Toray industries, Inc., Japan.

Electrodes of a droplet actuator are typically controlled by a controller or a processor, which is itself provided as part of a system, which may include processing functions as well as data and software storage and input and output capabilities.

Reagents may be provided on the droplet actuator in the droplet operations gap or in a reservoir fluidly coupled to the droplet operations gap. The reagents may be in liquid form, e.g., droplets, or they may be provided in a reconstitutable form in the droplet operations gap or in a reservoir fluidly coupled to the droplet operations gap. Reconstitutable reagents may typically be combined with liquids for reconstitution. An example of reconstitutable reagents suitable for use with the invention includes those described in Meathrel, et al., U.S. Pat. No. 7,727,466, entitled “Disintegratable films for diagnostic devices,” granted on Jun. 1, 2010.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing.

The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other.

The terms “splitting”, “separating”, and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more).

The term “mixing” refers to droplet operations which result in more homogeneous distribution of one or more components within a droplet.

Examples of “loading” droplet operations include microdialysis loading, pressure assisted loading, robotic loading, passive loading, and pipette loading.

Droplet operations may be electrode-mediated. In some cases, droplet operations are further facilitated by the use of hydrophilic and/or hydrophobic regions on surfaces and/or by physical obstacles. For examples of droplet operations, see the patents and patent applications cited above under the definition of “droplet actuator.” Impedance or capacitance sensing, or imaging techniques may sometimes be used to determine or confirm the outcome of a droplet operation. Examples of such techniques are described in Sturmer et al., International Patent Pub. No. WO/2008/101194, entitled “Capacitance Detection in a Droplet Actuator,” published on Aug. 21, 2008, the entire disclosure of which is incorporated herein by reference.

Generally speaking, the sensing or imaging techniques may be used to confirm the presence or absence or volume of a droplet at a specific electrode. For example, the presence of a dispensed droplet at the destination electrode following a droplet dispensing operation confirms that the droplet dispensing operation was effective.

Similarly, the presence of a droplet at a detection spot at an appropriate step in an assay protocol may confirm that a previous set of droplet operations has successfully produced a droplet for detection.

Droplet transport time can be quite fast. For example, in various embodiments, transport of a droplet from one electrode to the next may exceed about 1 sec, or about 0.1 sec, or about 0.01 sec, or about 0.001 sec.

In one embodiment, the electrode is operated in AC mode but is switched to DC mode for imaging. It is helpful for conducting droplet operations for the footprint area of the droplet to be similar to the electrowetting area; in other words, 1×-, 2×-3×-droplets are usefully controlled and operated using 1, 2, and 3 electrodes, respectively. If the droplet footprint is greater than the number of electrodes available for conducting a droplet operation at a given time, the difference between the droplet size and the number of electrodes should typically not be greater than 1; in other words, a 2× droplet is usefully controlled using 1 electrode and a 3× droplet is usefully controlled using 2 electrodes. When droplets include beads, it is useful for droplet size to be equal to the number of electrodes controlling the droplet, e.g., transporting the droplet.

“Filler fluid” means a fluid associated with a droplet operations substrate of a droplet actuator, which fluid is sufficiently immiscible with a droplet phase to render the droplet phase subject to electrode-mediated droplet operations. For example, the droplet operations gap of a droplet actuator is typically filled with a filler fluid. The filler fluid may, for example, be or include a low-viscosity oil, such as silicone oil or hexadecane filler fluid. The filler fluid may be or include a halogenated oil, such as a fluorinated or perfluorinated oil. The filler fluid may fill the entire gap of the droplet actuator or may coat one or more surfaces of the droplet actuator. Filler fluids may be conductive or non-conductive. Filler fluids may be selected to improve droplet operations and/or reduce loss of reagent or target substances from droplets, improve formation of microdroplets, reduce cross contamination between droplets, reduce contamination of droplet actuator surfaces, reduce degradation of droplet actuator materials, etc. For example, filler fluids may be selected for compatibility with droplet actuator materials.

As an example, fluorinated filler fluids may be usefully employed with fluorinated surface coatings. Fluorinated filler fluids are useful to reduce loss of lipophilic compounds, such as umbelliferone substrates like 6-hexadecanoylamido-4-methylumbelliferone substrates (e.g., for use in Krabbe, Niemann-Pick, or other assays); other umbelliferone substrates are described in U.S. Patent Pub. No. 20110118132, published on May 19, 2011, the entire disclosure of which is incorporated herein by reference. Examples of suitable fluorinated oils include those in the Galden line, such as Galden HT170 (bp=170° C., viscosity=1.8 cSt, density=1.77), Galden HT200 (bp=200 C, viscosity=2.4 cSt, d=1.79), Galden HT230 (bp=230 C, viscosity=4.4 cSt, d=1.82) (all from Solvay Solexis); those in the Novec line, such as Novec 7500 (bp=128 C, viscosity=0.8 cSt, d=1.61), Fluorinert FC-40 (bp=155° C., viscosity=1.8 cSt, d=1.85), Fluorinert FC-43 (bp=174° C., viscosity=2.5 cSt, d=1.86) (both from 3M).

In general, selection of perfluorinated filler fluids is based on kinematic viscosity (<7 cSt is preferred, but not required), and on boiling point (>150° C. is preferred, but not required, for use in DNA/RNA-based applications (PCR, etc.)).

Filler fluids may, for example, be doped with surfactants or other additives. For example, additives may be selected to improve droplet operations and/or reduce loss of reagent or target substances from droplets, formation of microdroplets, cross contamination between droplets, contamination of droplet actuator surfaces, degradation of droplet actuator materials, etc. Composition of the filler fluid, including surfactant doping, may be selected for performance with reagents used in the specific assay protocols and effective interaction or non-interaction with droplet actuator materials. Examples of filler fluids and filler fluid formulations suitable for use with the invention are provided in Srinivasan et al, International Patent Pub. Nos. WO/2010/027894, entitled “Droplet Actuators, Modified Fluids and Methods,” published on Mar. 11, 2010, and WO/2009/021173, entitled “Use of Additives for Enhancing Droplet Operations,” published on Feb. 12, 2009; Sista et al., International Patent Pub. No. WO/2008/098236, entitled “Droplet Actuator Devices and Methods Employing Magnetic Beads,” published on Aug. 14, 2008; and Monroe et al., U.S. Patent Publication No. 20080283414, entitled “Electrowetting Devices,” filed on May 17, 2007; the entire disclosures of which are incorporated herein by reference, as well as the other patents and patent applications cited herein. Fluorinated oils may in some cases be doped with fluorinated surfactants, e.g., Zonyl FSO-100 (Sigma-Aldrich) and/or others.

The terms “top,” “bottom,” “over,” “under,” and “on” are used throughout the description with reference to the relative positions of components of the droplet actuator, such as relative positions of top and bottom substrates of the droplet actuator. It will be appreciated that the droplet actuator is functional regardless of its orientation in space.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface or could be in contact with one or more layers or films that are interposed between the liquid and the electrode/array/matrix/surface. In one example, filler fluid can be considered as a film between such liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

### 6.2. Unbound Bilirubin Assay for Newborn-Screening Point-of-Birth Platform

The invention provides an apparatus and methods of performing a biochemical assay.

The invention provides a microfluidics device including droplets subject to manipulation by the device wherein the droplets comprise a surfactant of the invention. Preferably the droplets comprise blood. The droplets may be surrounded by a filler fluid, such as a low-viscosity oil, such as silicone oil. The device may be an electrowetting device, such as the devices described in International App. No. PCT/US08/72604, entitled “Use of additives for enhancing droplet operations”, which is incorporated herein by reference in its entirety.

In various aspects, the invention provides methods of performing a biochemical assay using a whole blood sample as input, wherein the whole blood sample can be processed into one or more fractions of the whole blood sample for analysis.

In one aspect, the whole blood sample is subjected to droplet operations on a microfluidics device.

In one aspect, the invention provides methods of performing a fluorescence-based biochemical assay.

In one aspect, the fluorescence-based biochemical assay is a newborn-screening assay for unbound bilirubin.

In one aspect, the fluorescence-based assay for unbound bilirubin uses a green fluorescent protein (GFP) that specifically binds to unbound/unconjugated bilirubin.

The methods of the invention can be processed, for example, on a point-of-birth system and instrument, such as the point-of-birth system and instruments described in International App. No. PCT/US17/30425, entitled “Point-of-birth system and instrument, biochemical cartridge, and methods for newborn screening”, which is incorporated herein by reference in its entirety.

### 6.2.1. Selection of Surfactants

The methods of the invention make use of surfactants.

In one aspect, a surfactant is selected for electrowetting a whole blood sample on a microfluidics cartridge without causing significant lysis of the whole blood sample.

Examples of surfactants suitable for electrowetting a whole blood sample on a microfluidics cartridge without causing significant lysis of the whole blood sample include: the non-ionic surfactants Tween® 80 (available from Millipore-Sigma, St. Louis, Missouri) having the structure:

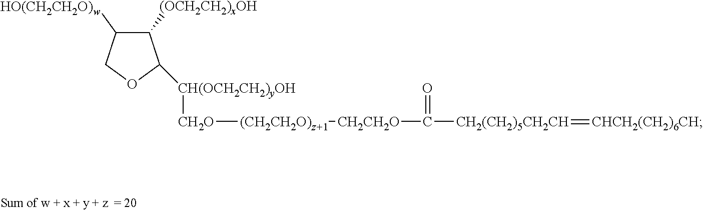

and Facade®-TEM (available from Avanti® Polar Lipids, Alabaster, Alabama) having the structure:

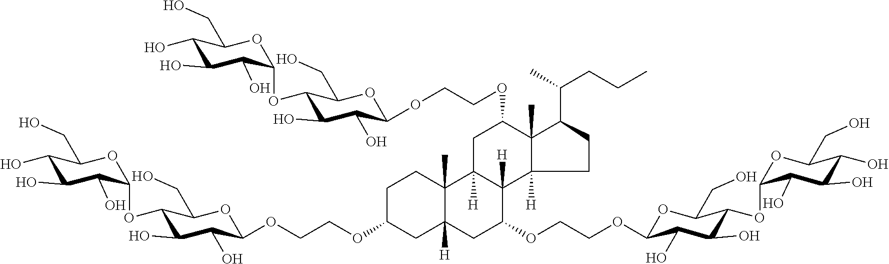

and zwitterionic surfactant 11:0 Lyso PC (available from Avanti® Polar Lipids, Alabaster, Alabama) having the structure:

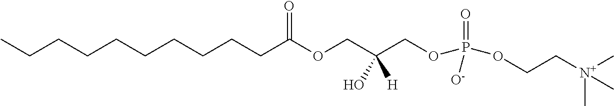 [SMILES: CCCCCCCCCCC(=O)OC[C@@H](O)COP(=O)([O-])OCC[N+](C)(C)C]

In one aspect, a surfactant is selected for performing a fluorescence-based assay on a microfluidics cartridge without interfering with a fluorescence signal.

In one aspect, a surfactant is selected for conducting a fluorescence-based assay using a green fluorescent protein (GFP) that specifically binds to unbound/unconjugated bilirubin, such as the protein UnaG described in Kumagai, A., et. al., Cell (2013) 153:1602-1611, which is incorporated herein by reference in its entirety.

### 6.2.2. Microfluidic Device and Methods

The invention provides a microfluidics device including droplets subject to manipulation by the device wherein the droplets comprise a surfactant of the invention.

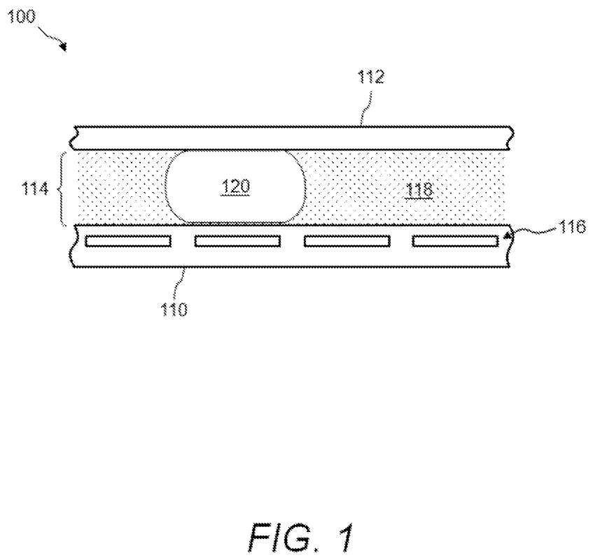

FIG. 1 is a cross-sectional view illustrating an example of a portion of a microfluidics device 100 for performing a biochemical assay in droplets. Microfluidics device 100 includes a bottom substrate 110 and a top substrate 112 separated by a gap 114. A set of droplet operations electrodes 116, e.g., electrowetting electrodes, are arranged, for example, on bottom substrate 110. The droplet operations electrodes 116 are arranged for conducting droplet operations, such as droplet loading, dispensing, splitting, transporting, merging, and mixing. Gap 114 is filled with a filler fluid 118. Filler fluid 118 may, for example, be a low-viscosity oil, such as silicone oil.

An aqueous droplet 120 may be present in gap 114 of microfluidics device 100. In one example, droplet 120 is a droplet of sample fluid to be evaluated, such as a whole blood droplet or a blood component droplet, or a droplet including blood cells, or a droplet including red blood cells. In another example, droplet 120 is a reagent droplet for conducting a biochemical assay, such as an enzyme droplet, or a stop solution droplet, or dilution buffer droplet, or detection reagent droplet. Oil filler fluid 118 fills gap 114 and surrounds droplet 120.

Droplet 120 includes a surfactant that is suitable for conducting a biochemical assay using electrowetting to conduct droplet operations. In one example, the surfactant is suitable for conducting a blood-based assay without significant lysis of red blood cells. In one example, the surfactant is suitable for conducting an enzymatic, fluorescence-based biochemical assay. In one example, the surfactant is Tween® 80.

6.2.3. GOD-POD-UnaG Assay

The invention provides systems, cartridges and methods for measuring unbound bilirubin on a microfluidic device using a fluorescence-based biochemical assay.

The invention makes use of enzyme-mediated oxidative decomposition of unbound bilirubin (i.e., conjugated and unconjugated bilirubin that is not bound to albumin) and UnaG specific binding to measure the levels of unbound (unconjugated) bilirubin in a whole blood sample. The International App. No. PCT/JP2016/060327, entitled “Measurement method for unbound bilirubin in blood sample”, which is incorporated herein by reference in its entirety, describes a method for using glucose oxidase (GOD) and peroxidase (POD) in combination with UnaG to measure unbound bilirubin in a blood sample. The GOD-POD-UnaG assay includes: (1) a decomposition step using a GOD-POD reaction to oxidize unbound bilirubin; (2) a “stop” decomposition step to stop the decomposition reaction and give a decomposition product; (3) a “contact” step to contact the decomposition product (of step 1) and an unreacted sample (i.e., blood sample not subjected to decomposition step 1) with UnaG that binds unconjugated bilirubin; and (4) a detection step wherein the fluorescence of UnaG is detected from the decomposition product sample and from the unreacted sample. The amount of unbound bilirubin is determined from the difference between the detected values.

The methods of the invention use a microfluidic device that includes an arrangement of droplet operations electrodes that are configured for conducting a GOD-POD-UnaG assay for unbound bilirubin. In one aspect, all assay reagents (e.g., diluent, buffer, enzyme reagents, stop reagent, UnaG detection reagent) for conducting a GOD-POD-UnaG assay are provided “pre-loaded” on the microfluidic device.

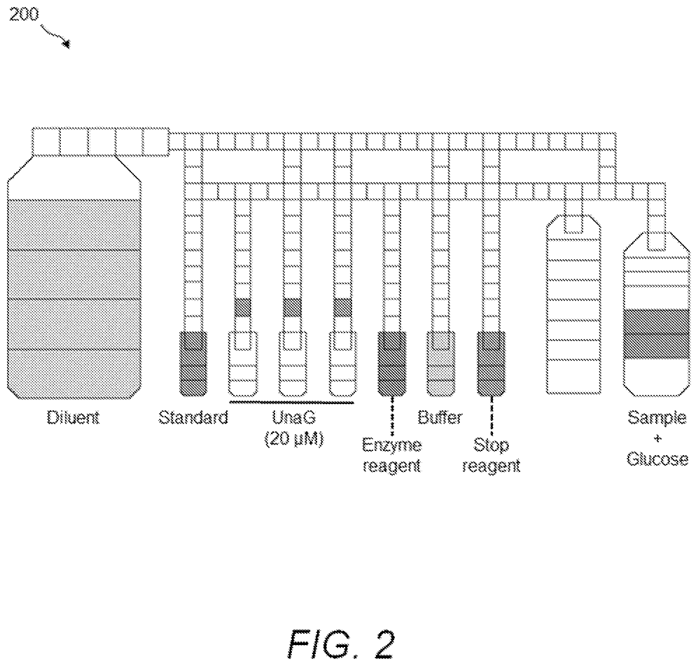

FIG. 2 is a schematic diagram 200 illustrating an example of an arrangement of droplet operations electrodes configured for conducting a GOD-POD-UnaG assay for unbound bilirubin on a microfluidic device. The arrangement of droplet operations electrodes includes electrode reservoirs for dispensing a diluent solution, a fluorescence standard solution, an enzyme reagent solution (e.g., GOD and POD solution), a buffer solution, a stop reagent (e.g., ascorbic acid); one or more assay reaction zones; and a sample reservoir for loading and diluting a blood sample. The arrangement of droplet operations electrodes also includes detection reagent electrodes whereon the UnaG detection reagent is dried.

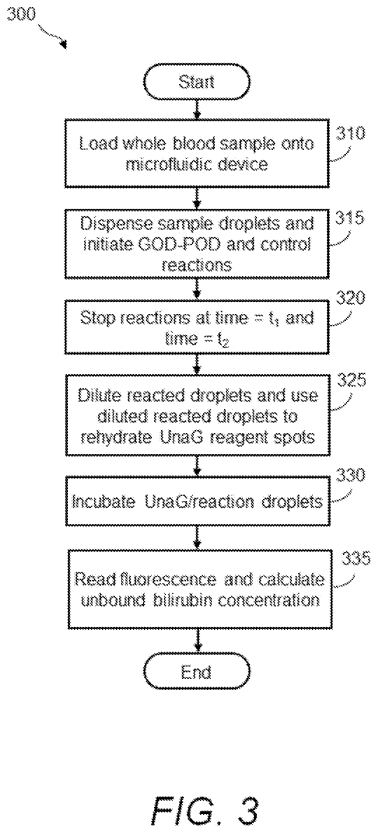

FIG. 3 is a flow diagram illustrating an example of a method 300 for measuring unbound bilirubin in a blood sample using a GOD-POD-UnaG assay on a microfluidic device. Method 300 may include any or all of the following steps as well as additional unspecified steps.

At a step 310, a sample is loaded onto a microfluidic device. For example, a 50 μL of a whole blood sample is loaded into a sample reservoir of the microfluidic device and diluted in glucose buffer (e.g., diluted 1:27 in glucose buffer).

At a step 315, sample droplets are dispensed, and GOD-POD and control reactions are initiated. For example, a first sample droplet is dispensed and combined with a buffer droplet to yield a control reaction droplet (sample dilution 1:54); a second sample droplet is dispensed and combined with an enzyme reagent droplet to yield a “short” reaction droplet (sample dilution 1:54); and a third sample droplet is dispensed and combined with an enzyme reagent droplet to yield a “long” reaction droplet (sample dilution 1:54). The GOD-POD enzyme reagent oxidizes the sample at a rate dependent on the amount of unbound bilirubin in the sample.

At a step 320, the GOD-POD and control reactions are stopped at time=t1 and time=t2. For example, at a first time point t1=48 seconds, a stop reagent droplet is dispensed and combined with the control reaction droplet to yield control reacted droplet; a second stop reagent droplet is dispensed and combined with the “short” enzyme reaction droplet to yield a “short” reacted droplet. At time point t=120 seconds, a stop reagent droplet is dispensed and combined with the “long” enzyme reaction droplet to yield a “long” reacted droplet.

At a step 325, the reacted sample droplets are diluted and used to rehydrate UnaG reagent spots. For example, the control reacted droplet, the “short” enzyme reacted droplet, and the “long” enzyme reacted droplet are diluted 1:324. Each diluted droplet is then used to rehydrate a dried UnaG reagent spot.

At a step 330, the UnaG/reaction droplets are incubated. For example, the control reacted droplet is incubated with UnaG reagent to measure all unconjugated bilirubin (i.e., unbound+albumin bound). The “short” enzyme reacted droplet and the “long” enzyme reacted droplet are incubated with UnaG reagent to measure remaining unconjugated bilirubin.

At a step 335, fluorescence is read, and the amount of unbound bilirubin is calculated.

### 6.3. Examples

A surfactant screening protocol was used to identify one or more surfactants that are compatible with performing a UnaG-based fluorescence assay for bilirubin on an electrowetting fluidics cartridge. The screening protocol started with a panel of 96 different surfactants and proceeded stepwise to assess fluorescence interference, hemolysis of a whole blood sample, and electrowetting compatibility and limits, wherein each subsequent step used a downselected set of the original surfactant panel.

### 6.3.1. Fluorescence Interference

The surfactant used in electrowetting a sample and/or reaction droplet in an on-cartridge assay can interfere with the fluorescence signal generated by UnaG binding to bilirubin. To assess the effect of different surfactants on bilirubin-induced UnaG fluorescence, an on-bench microtiter plate assay was performed.

For this assay, 96 different detergents from a Detergent Screen™ kit (available from Hampton Research; Aliso Viejo, CA) were used. The detergents in the kit included ionic (n=13), non-ionic (n=46), and zwitterionic detergents (n=31), and non-ionic sulfobetaines (n=6). All detergents were used at either 0.5× critical micelle concentration (CMC) or at a dilution of 1:20 of the provided stock for non-micelle forming surfactants. The bilirubin sample used was a single total bilirubin (TBIL) sample that was known to have significant fluorescence interference from 0.1% Tween® 20 in a UnaG binding reaction. Test detergents were only added to aliquots of the test sample and not to the UnaG reagent.

Briefly, the test TBIL sample was diluted 1:9 in a glucose buffer (25-100 mM phosphate buffer at pH 7.4 with 1 mg/mL glucose). Each detergent was diluted to a 2× stock solution in Dulbecco's phosphate buffered saline (i.e., CMC or 1:10 from provided stock). In individual microtiter plate wells, 10 μL of diluted sample and 10 μL of 2× stock detergent were combined (i.e., mixed 1:1) and incubated at room temperature for 7 minutes. To account for the amount of time it takes to pipette across a microtiter plate(s), a no-detergent control was included in each column of the microtiter plate(s). At the end of the incubation period, 20 μL of UnaG (20 μM in phosphate buffer without detergent) was added to each well and fluorescence was immediately read at room temperature (24.4°) and Ex/Em of 488/528 nm. For data analysis, the percent bias (% bias; detergent containing sample compared to no-detergent control) of relative fluorescence units (RFU) was compared at t=3 minutes and a cut-off was set at ±10%.

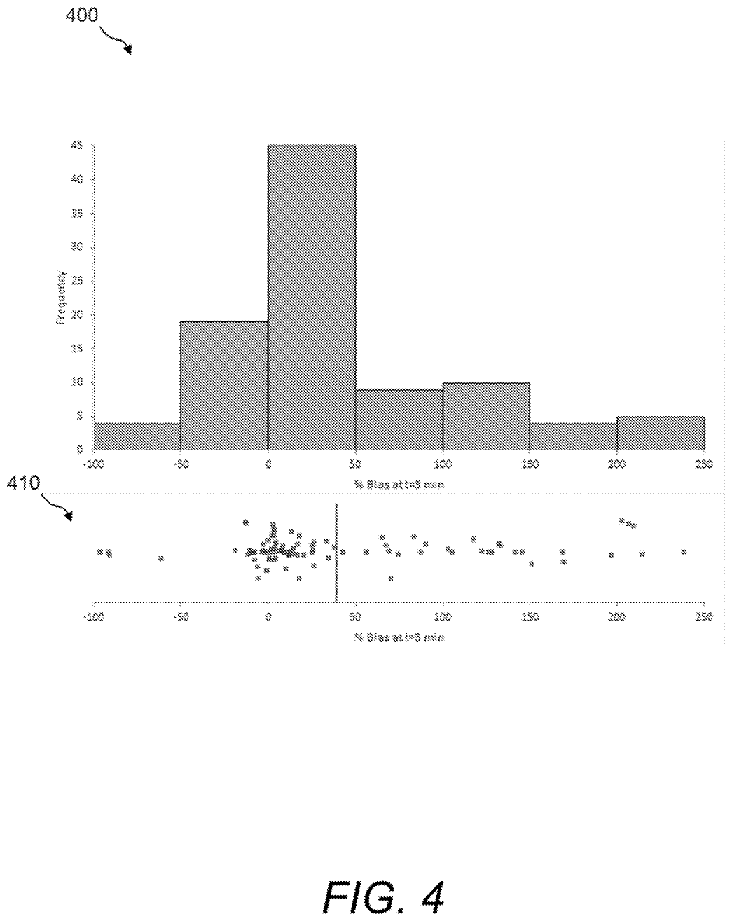

FIG. 4 is a plot 400 and a plot 410 showing the % bias of RFUs at t=3 minutes for the 96 detergents tested. The data show that detergents were more likely to increase rather than decrease the fluorescence signal, an observation that is consistent with previous observations.

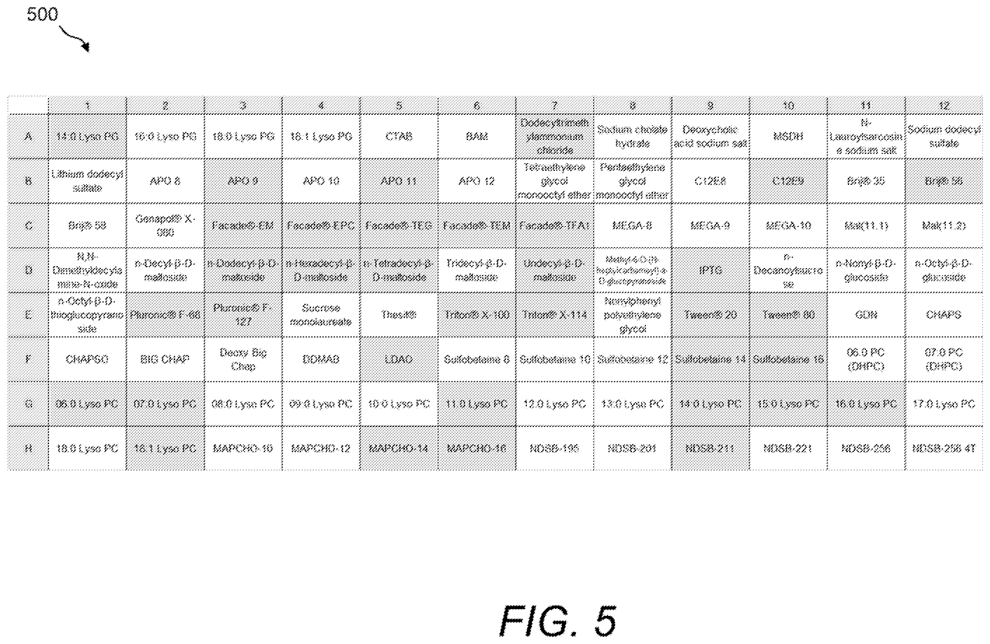

FIG. 5 is a table 500 showing the microtiter plate layout of the 96 detergents in the Detergent Screen™ kit used to screen for interference in the UnaG-bilirubin fluorescence assay. Cells shaded in gray indicate a downselected set of 35 detergents that had <10% bias at t=3 minutes. The downselected set of n=35 surfactants includes: 14:0 Lyso PG, Dodecyltrimethylammonium chloride, APO 9, APO 11, C₁₂E₉, Brij® 56, Facade®-EM, Facade®-EPC, Facade®-TEG, Facade®-TEM, Facade®-TFA1, n-Dodecyl-β-D-maltoside, n-Hexadecyl-β-D-maltoside, n-Tetradecyl-β-D-maltoside, Undecyl-β-D-maltoside, IPTG, Pluronic® F-68, Pluronic® F-127, Triton® X-100, Triton® X-114, Tween® 20, Tween® 80, LDAO, Sulfobetaine 14, Sulfobetaine 16, 06:0 Lyso PC, 07:0 Lyso PC, 11:0 Lyso PC, 14:0 Lyso PC, 15:0 Lyso PC, 16:0 Lyso PC, 18:1 Lyso PC, MAPCHO-14, MAPCHO-16, and NDSB-211.

### 6.3.2. Hemolysis of Whole Blood Sample

In a multiplexed on-cartridge assay for newborn screening, a whole blood sample is typically split on-cartridge into two or more aliquots for performing different biochemical tests. One set of tests (e.g., albumin or bilirubin) may be performed using a plasma fraction of the whole blood sample (i.e., the whole blood sample is agglutinated on-cartridge to yield a plasma sample) and a second set of tests (e.g., hemoglobin or glucose-6-phosphate dehydrogenase (G6PD)) may be performed using a lysed whole blood sample (i.e., the whole blood sample is lysed on-cartridge).

To determine whether a surfactant can induce hemolysis of a whole blood sample, an on-bench microtiter plate assay was performed. For this assay, the downselected set of n=35 surfactants from the fluorescence interference assay was used. All surfactants were at 20× desired test concentration (i.e., 0.5×CMC or 1:20 for non-micelle forming surfactants; 21 surfactants were at 20× in the original plate stock; 14 surfactants were diluted and vortexed before use).

Briefly, 4 μL of each 20× surfactants stock were added to individual microtubes that contained 76 μL of Dulbecco's phosphate buffered saline (DPBS). Aliquots (80 μL) of a whole blood sample were then added to each microtube and the surfactants-whole blood solution was mixed by flicking/inverting the microtubes. The samples were incubated at room temperature for 10 minutes, with additional mixing 2× during the incubation period. At the end of the incubation period, the microtubes were centrifuged at 2500 RPM for 5 minutes. An aliquot (40 μL) of each supernatant was removed and transferred to a 96-well half area plate. The plates were scanned, and optical density (absorbance) measurements were obtained for hemoglobin. The amount of hemoglobin was quantified as gHb/L using Beer's law and the following: L=0.183 cm; E=53,236 L/cm*mol; MW=64,500 g/mol; DF=3 (assuming a ˜50% HCT sample). A cut-off for inducing hemolysis was set at a >1.5-fold change from control.

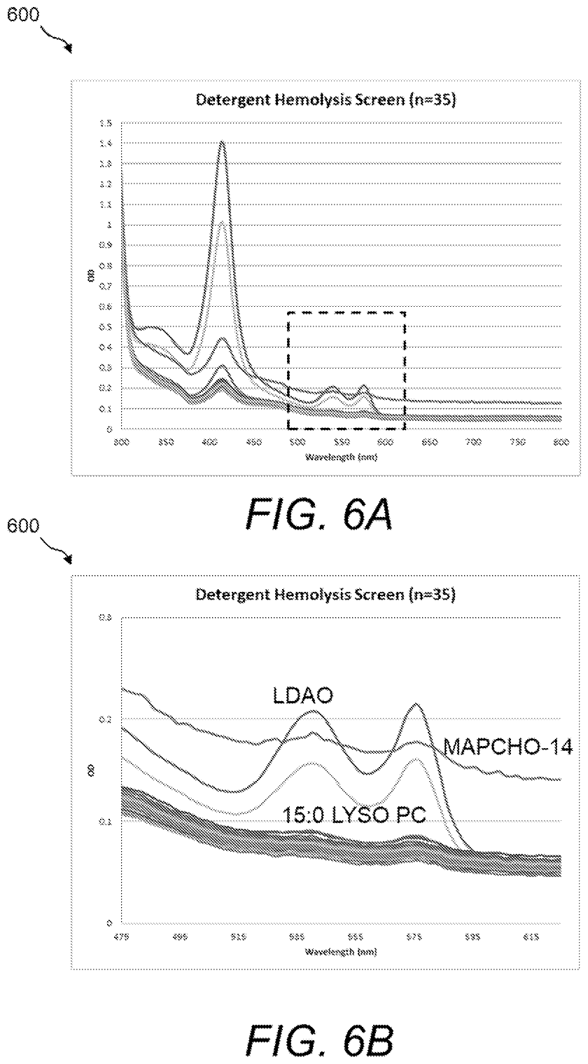

FIG. 6A and FIG. 6B are plots 600 showing a spectral scan for hemoglobin in the n=35 surfactant samples. FIG. 6B is a close-up of the boxed region of plot 600 of FIG. 6A. The data show that 3 surfactant samples (LDAO, MAPCHO-14, and 15:0 LYSO PC; see FIG. 6B) caused significant hemolysis, while the other 32 surfactants did not.

### 6.3.3. Electrowetting Compatibility and Limits

To qualitatively assess compatibility with electrowetting, the downselected set of n=35 surfactants were tested on FINDER® cartridges (available from Baebies, Inc., Durham, NC). All surfactants were tested at 0.5×CMC or 1:20 for non-micelle forming surfactants. For this assessment, 160 μL of a surfactant was loaded in the diluent reservoir of a FINDER® cartridge, and the cartridge was then placed in a FINDER® instrument (available from Baebies, Inc., Durham, NC). A Panel 1 script was run on the FINDER® instrument to assess surfactant/diluent priming and dispensing of each surfactant. The electrowetting operations were visually assessed and graded on a letter scale as follows: A+=desired; A and A⁻=acceptable for next round screening. The assessment included observations such as how the surfactant/diluent loaded into the reservoir, how quickly the surfactant/diluent solution moved up the reservoir to dispensing electrodes, dispensing out of the reservoir, and number of electrodes traversed.

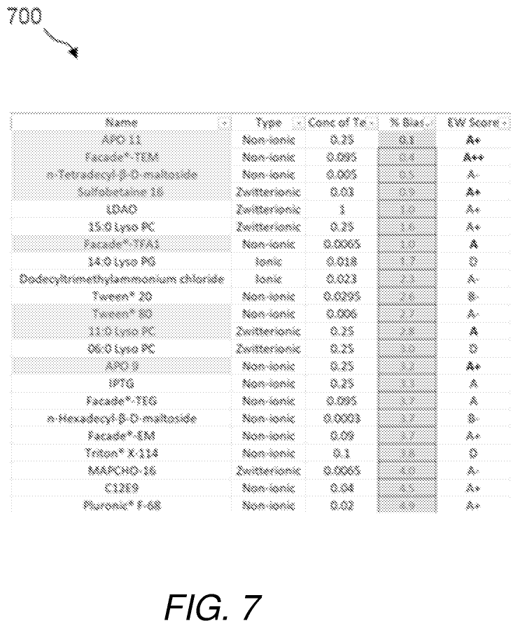

FIG. 7 is a screenshot 700 showing the data sheet for the priming/dispensing assessment of some of the n=35 downselected surfactants. For each surfactant listed, the data sheet shows the concentration tested, the electrowetting score (EW Score) and the observation notes. For each surfactant, the data sheet also lists the percent bias (% Bias) from the initial fluorescence interference screen described with reference to FIG. 4. The electrowetting observations and the percent bias were used to select a second downselected surfactant set of n=8 (APO9, APO 11, Facade®-TEM, Facade®-TFA1, n-Tetradecyl-β-D-maltoside, Tween®80, Sulfobetaine 16, and 11:0 Lyso PC) that show the best performance (i.e., least % bias, “A” electrowetting score, no fluorescence interference, and no hemolysis).

A second electrowetting screen was performed to define the lowest surfactant concentration that electrowets with an “A+” score. For this assessment, the second downselected set of n=8 surfactants were used. In this electrowetting screen, the concentration of a surfactant previously scored in FIG. 7 as “A” or “A−” was increased 4×; and the concentration of a surfactant previously scored as “A+” was reduced 10×.

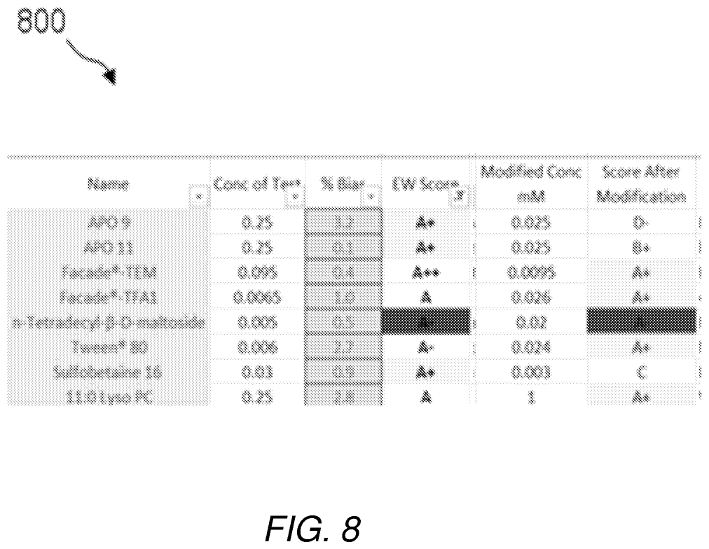

FIG. 8 is a screenshot 800 showing the data sheet for the electrowetting assessment of the second downselected set of n=8 surfactants using modified surfactant concentrations. Visual observations for electrowetting operations prime/dispense were made as described with reference to FIG. 7. For each surfactant listed, the data sheet shows the concentration tested, the electrowetting score (EW Score) and the observation notes from the original assessment and from the assessment using modified surfactant concentrations. The data sheet also lists the % Bias from the initial fluorescence interference screen described with reference to FIG. 4. From this assessment, one surfactant (n-Tetradecyl-13-D-maltoside) could not be brought to the “A+” stage using a 4× increase concentration. The electrowetting observations using modified surfactant concentrations were used to select a third downselected surfactant set of n=7 (APO9, APO 11, Facade®-TEM, Facade®-TFA1, Tween® 80, Sulfobetaine 16, and 11:0 Lyso PC).

### 6.3.4. Sample Bias (Variability) in UnaG+Bilirubin Fluorescence Assay

To assess the effect of the third downselected set of n=7 set surfactants (APO9, APO 11, Facade®-TEM, Facade®-TFA1, Tween® 80, Sulfobetaine 16, and 11:0 Lyso PC) on sample-to-sample variability in bilirubin-induced UnaG fluorescence, an on-bench microtiter plate assay was performed using plasma samples (n=12 plasma samples) with known TBIL concentrations spanning a range from 3.5 to 23.7 mg/dL.

Briefly, all surfactants were adjusted to 2× desired test concentration in the glucose buffer. The plasma samples (n=12) were diluted 1:9 in the glucose buffer without detergent. UnaG was diluted to 20 μM in a phosphate buffer without detergent. Aliquots (10 μL) of diluted plasma samples were loaded into designated wells of a microtiter plate (n=8 wells per column: 12 columns for n=12 plasma samples). Using a multichannel pipette, 10 μL aliquots of a detergent was dispensed across a row of the n=12 plasma samples (i.e., 1 row for each n=7 detergents and 1 row for a glucose buffer control). The plate was incubated at room temperature with shaking for 5 minutes. At the end of the incubation period, 20 μL of UnaG was added to each well and kinetic fluorescence was read. For data analysis, the percent bias (% bias; detergent containing sample compared to glucose buffer only control) was compared at t=3 minutes.

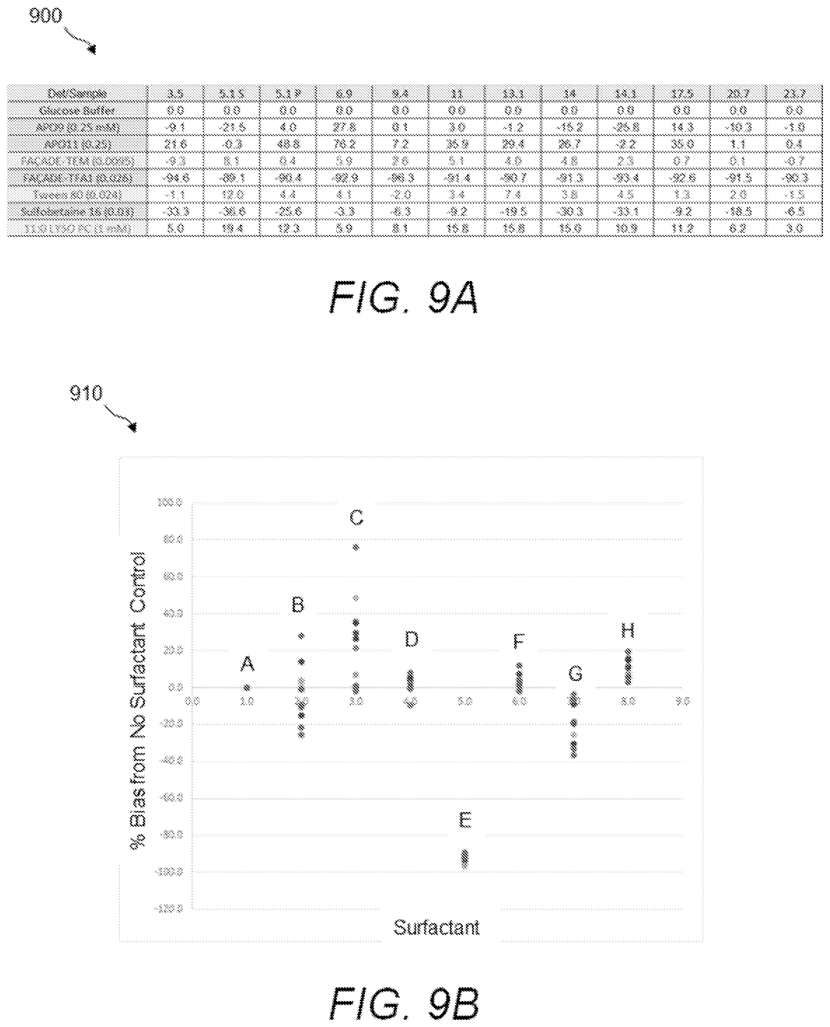

FIG. 9A is a table 900 showing the percent bias from control for the 7 surfactants and 12 plasma samples used to screen for fluorescence interference. The top row (shaded in gray) of table 900 is the TBIL value for each sample tested (i.e., 3.5=TBIL value for plasma sample 1; 5.1S=TBIL value for plasma sample 2; etc.). The first column of table 900 shows the no surfactant control (“Glucose buffer”) and the 7 detergents (APO 9, APO 11, Facade®-TEM, Facade®-TFA1, Tween® 80, Sulfobetaine 16, and 11:0 Lyso PC) and concentrations (in parenthesis) used in the assay.

FIG. 9B is a plot 910 showing percent bias from the no surfactant control at t=3 minutes for the 7 surfactants tested. The data points are the 12 different plasma samples, and the “columns” of data points are: A=no surfactant control (glucose buffer only), B=APO9, C=APO11, D=Facade®-TEM, E=Facade®-TFA1, F=Tween® 80, G=Sulfobetaine 16, and H=11:0 Lyso PC. The data show that at t=3 minutes, all Facade®-TEM samples and all but one Tween® 80 sample are within 10% of the no surfactant control. One of the surfactants (Facade®-TFA1) used at the increased concentration with an A+ electrowetting (FIG. 7, modified concentration) now interferes with fluorescence. Three surfactants (APO9, APO11, and Sulfobetaine 16) had very noisy signals across the different plasma samples (i.e., sample to sample variability; an observation previously noted for Tween® 20). Three of the surfactants (Facade®-TEM, Tween® 80, and 11:0 Lyso PC) were consistent across all 12 plasma samples tested. Consistency in percent bias across all plasma samples was used to select a fourth downselected surfactant set of n=3 (Facade®-TEM, Tween® 80, and 11:0 Lyso PC).

The effect of Facade®-TEM, Tween® 80, and 11:0 Lyso PC surfactants on bilirubin-induced UnaG fluorescence was further assessed using six additional assayed plasma samples with known TBIL concentrations spanning a range from 1.2 to 10 mg/dL. Four plasma samples (i.e., 6.9, 13.1, 14, and 23.7) were used previously in FIG. 8. In this example, replicates of the control (no surfactant) and Facade®-TEM reactions were used for all plasma samples.

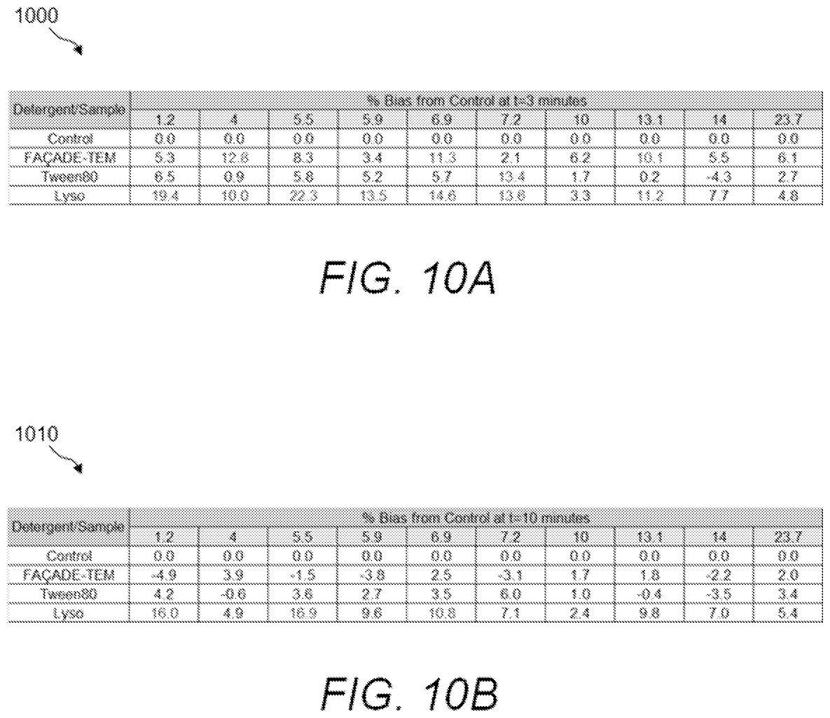

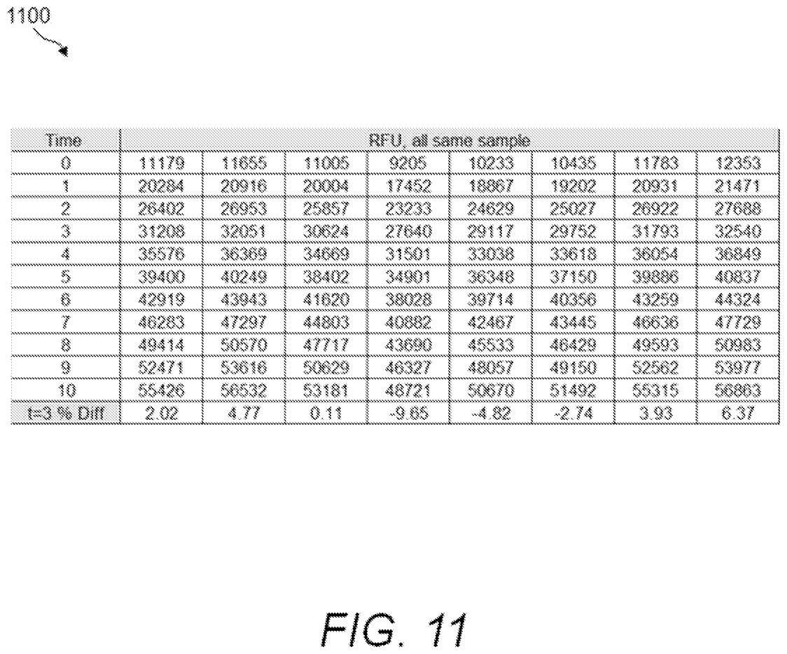

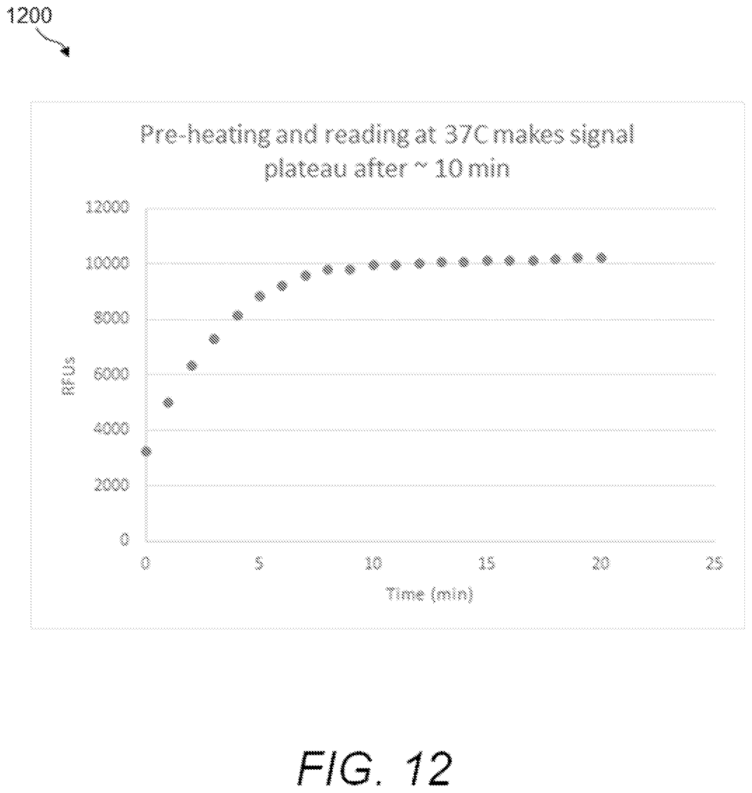

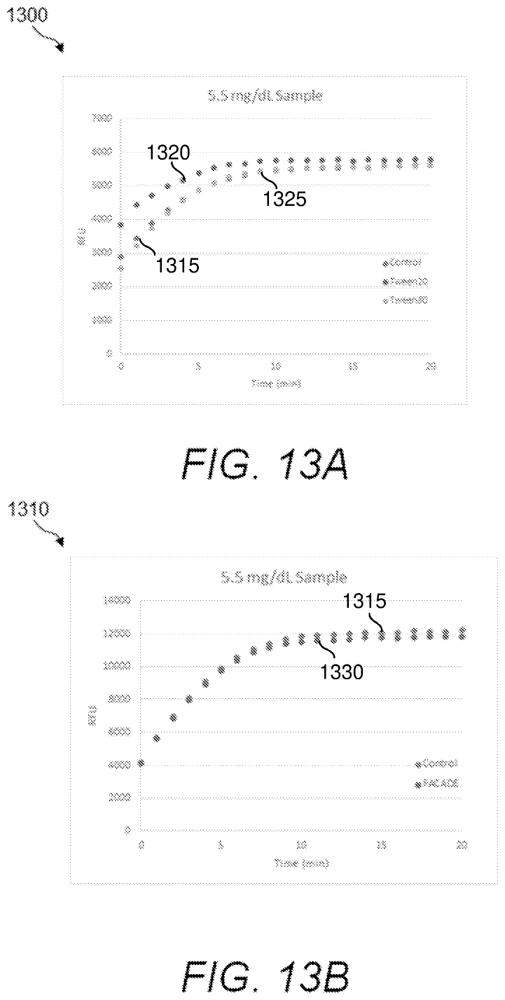

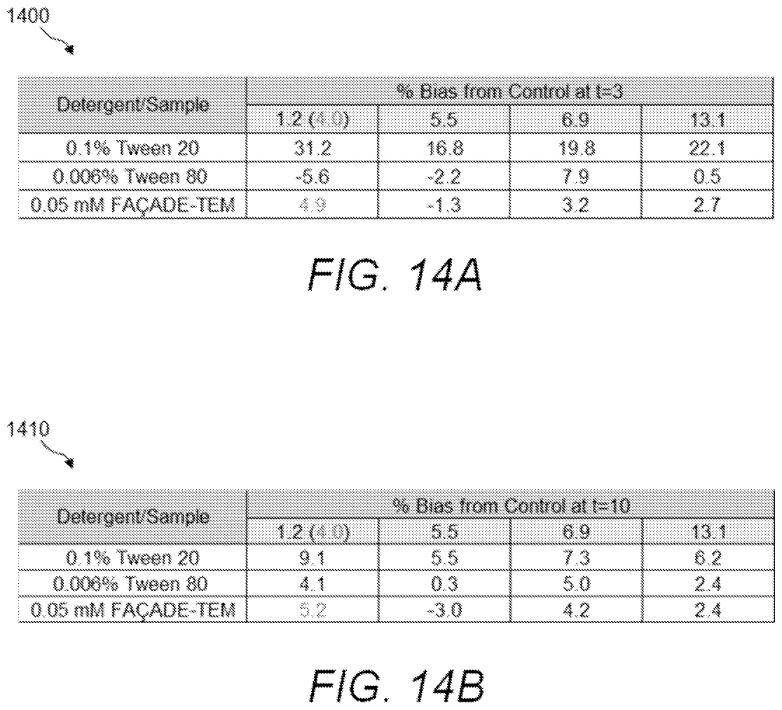

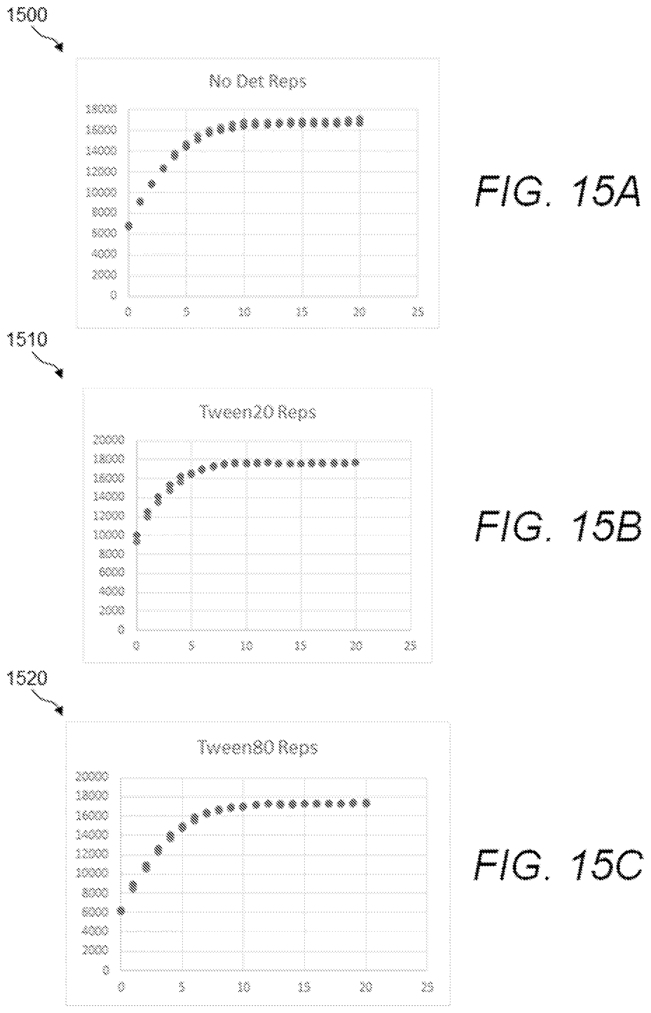

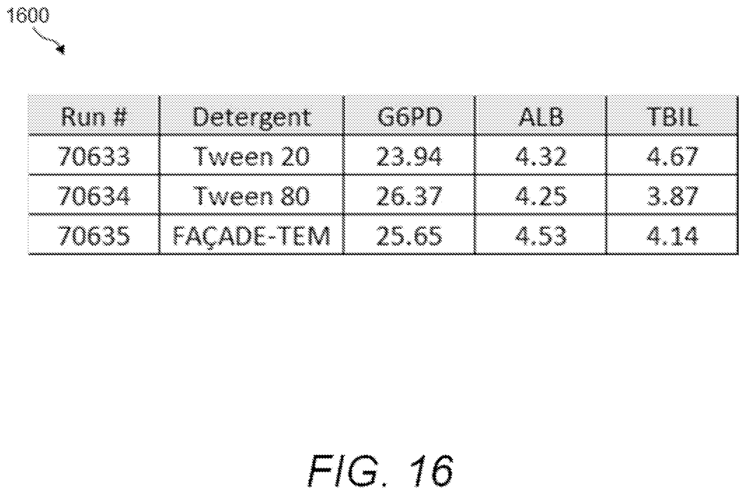

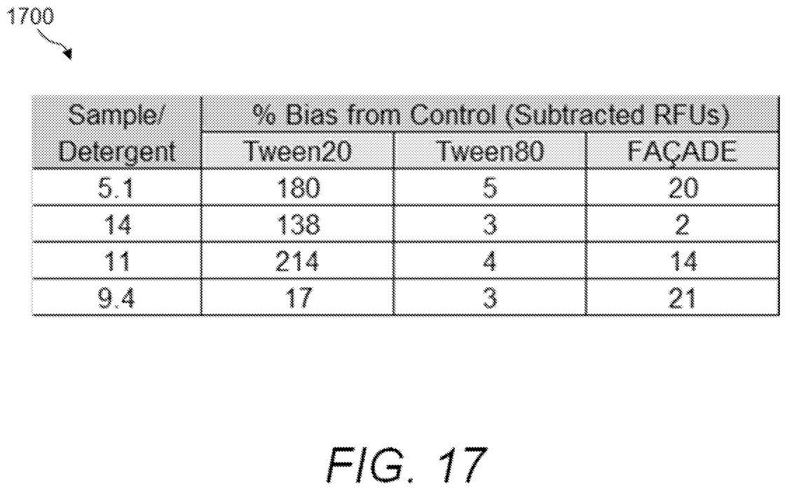

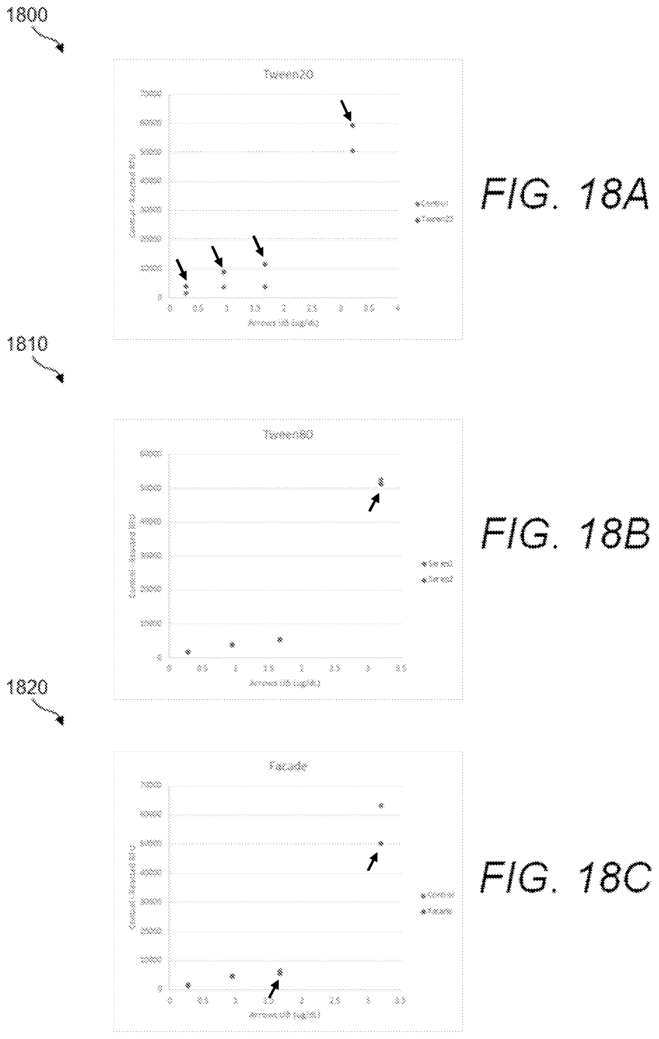

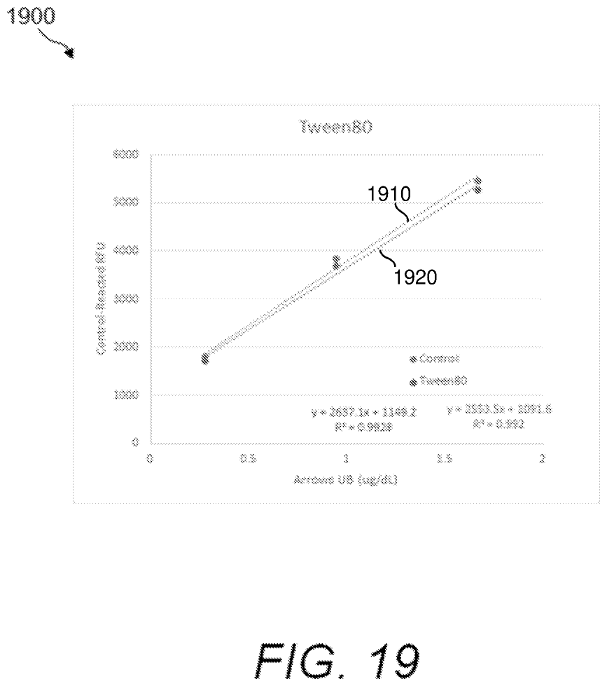

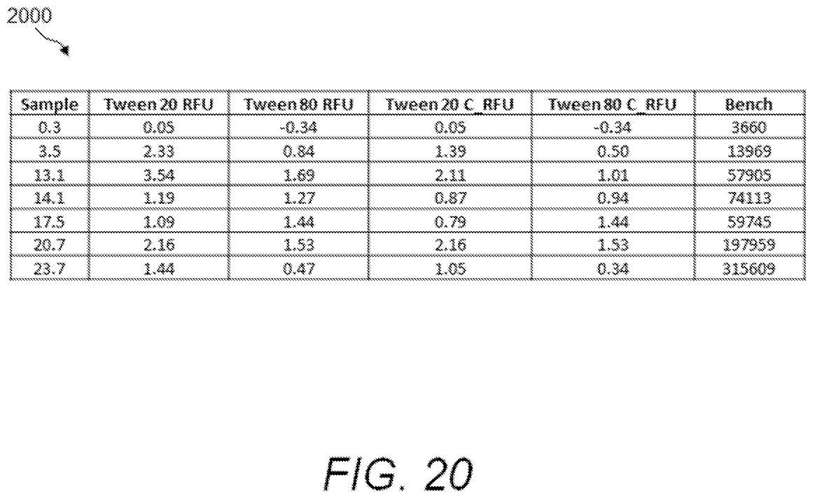

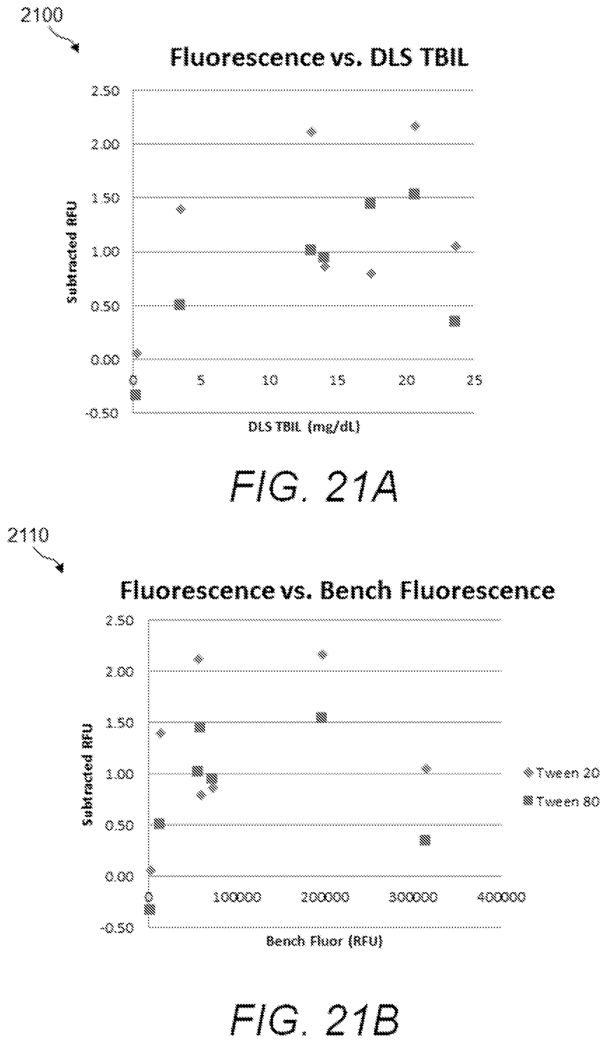

FIG. 10A is a table 1000 showing the percent bias from control at t=3 minutes for the 3 surfactants and 10 plasma samples used to screen for fluorescence interference. The top row (shaded in gray) of table 1000 is the TBIL value for each sample tested (i.e., 1.2=TBIL value for plasma sample 1; 4=TBIL value for plasma sample 2; etc.). The first column of table 1000 shows the control (glucose buffer) and the 3 detergents (Facade®-TEM, Tween®80, and 11:0 Lyso PC). The percent bias from control data for the control and Facade®-TEM reactions is the average of the replicate values. The data show that at t=3 minutes all but one Tween® 80 sample is within 10% of control.

FIG. 10B is a table 1010 showing the percent bias from control at t=10 minutes for the 3 surfactants and 10 plasma samples used to screen for fluorescence interference. The data show that at t=10 minutes all Tween® 80 and Facade®-TEM samples are within 6% of control.

Some noise was observed across the replicates for the control samples and the Facade®-TEM samples. It was also observed that the percent bias from control was decreased at t=10 minutes compared to percent bias from control at t=3 minutes. To investigate this variability (noise), the on-bench fluorescence interference assay was performed using replicates (n=8) of a single sample.

FIG. 11 is a table 1100 showing RFUs over time for the on-bench fluorescence interference assay used to assess variability. The coefficient of variation (CV) for the fluorescence interference assay was determined to be CV=5.4% (across n=8 replicates) and had a range of −9.7% to +6.4% bias from average.

Variation in the on-bench fluorescence assay could be due to mixing/pipetting methods used (e.g., multichannel pipetting), insufficient UnaG reagent (e.g., insufficient UnaG for higher levels of TBIL), reaction volume (relative to path length for signal detection), and/or fluorescence read time and temperature (e.g., room temperature vs. 37° C.). To address these possibilities several changes to the on-bench protocol were made: (1) sample dilution was increased from 1:36 to 1:240 (or 1:120) while leaving the concentration of UnaG at 10 μM; (2) individual sample and surfactant solutions were prepared in microtubes and then loaded into microtiter plate wells (eliminating the use of multichannel pipetting); (3) the final reaction volume was increased from 40 μL to 50 μL in the half-area microtiter plate (providing a 25% longer path length); (4) sample/surfactant solutions and UnaG reagent were pre-warmed to 37° C. prior to initiating the binding reaction; and (5) read time was increased from 10 minutes to 20 minutes.

FIG. 12 is a plot 1200 showing an example of a UnaG-bilirubin binding assay performed using the modified fluorescence interference assay.

### 6.3.5. Electrowetting Refinement for Facade®-TEM, Tween® 80, and 11:0 Lyso PC

An electrowetting screen was performed to further define the lowest surfactant concentration that could be used in a fluorescence interference assay and still maintain efficient electrowetting. Previous electrowetting screens (described with reference to FIG. 7 and FIG. 8) used Facade®-TEM at 0.095 mM and 0.0095 mM; Tween® 80 at 0.006 mM and 0.024 mM; and 11:0 Lyso PC at 0.25 mM and 1 mM.

For this electrowetting screen, stock solutions of Facade®-TEM (available from Avanti® Polar Lipids, Alabaster, Alabama); Tween® 80 (available from Millipore-Sigma, St. Louis, Missouri); and 11:0 Lyso PC (available from Avanti® Polar Lipids, Alabaster, Alabama) were made. Adjusted concentrations 2-3× above and below the previously tested concentrations were used. The electrowetting screen was performed to define the lowest surfactant concentration that electrowets with an “A+” or borderline “A” score.

To qualitatively assess compatibility with electrowetting, the downselected set of n=3 surfactants were tested on FINDER® cartridges (available from Baebies, Inc., Durham, NC). For this assessment, 160 μL of a surfactant was loaded in the diluent reservoir of a FINDER® cartridge, and the cartridge was then placed in a FINDER® instrument. A Panel 1 script was run on the FINDER® instrument to assess surfactant/diluent priming and dispensing of each surfactant individually. The electrowetting operations were visually assessed (as described with reference to FIG. 7) and graded on a letter scale as follows: A+=desired; A and A⁻=acceptable for next round screening.

From this assessment, it was determined that in-house made stocks of 0.0475 mM Facade®-TEM, 0.045 mM Tween® 80 (or 0.006%), and 11:0 Lyso PC can be used for efficient electrowetting in the fluorescence interference assays.

### 6.3.6. Retesting Facade®-TEM and Tween® 80 for Sample Bias (Variability) Using Updated UnaG Fluorescence Protocol

The updated UnaG fluorescence protocol described with reference to FIG. 10 and FIG. 11 was used to retest Facade®-TEM and Tween® 80 for sample-to-sample variability using plasma samples (n=4) with known TBIL concentrations spanning a range from 1.2 to 13.1 mg/dL (available from Discovery Life Sciences).

Briefly, plasma samples (n=4) were diluted 1:240 in glucose buffer with or without final surfactant (i.e., control samples) concentrations of 0.006% Tween® 80 or 0.05-mM Facade®-TEM or 0.1% Tween® 20 (used as positive interference control). UnaG was diluted to 20 μM in a phosphate buffer with no surfactant. Samples and the UnaG reagent were pre-warmed to 37° C. Samples were then loaded individually into separate wells of a microtiter plate in replicates of n=2 for each sample. UnaG was then aliquoted using a multichannel pipette into each sample well and fluorescence read at t=3 minutes and t=10 minutes.

FIG. 13A is a plot 1300 showing the RFU over time in reactions using the 5.5 mg/dL TBIL plasma sample and the surfactants Tween® 20 or Tween® 80. The data show that at the curve plateau Tween® 80 samples (plot line 1325) were within about 5% of control values (plot line 1315) and Tween® 20 samples (plot line 1320) were within about 9% of control. The data also show that Tween® 20 interference is more pronounced at earlier time points as evidenced by changes in the shape of the data curve.

FIG. 132B is a plot 1310 showing the RFU over time in reactions using the 5.5 mg/dL TBIL plasma sample and the surfactant Facade®-TEM. The data show that at the curve plateau Facade®-TEM samples (plot line 1330) were within about 5% of control values (plot line 1315).

FIG. 14A is a table 1400 showing the percent bias from control at t=3 minutes for Tween® 80, Facade®-TEM, and Tween® 20 in the UnaG fluorescence assay. The top row (shaded in gray) of table 1400 is the TBIL value for each sample tested (i.e., 1.2 (4.0)=TBIL value for plasma sample 1; 5.5=TBIL value for plasma sample 2; etc.). The percent bias from control data (i.e., no surfactant) for the reactions is the average of the replicate values.

FIG. 14B is a table 1410 showing the percent bias from control at t=10 minutes for Tween® 80, Facade®-TEM, and Tween® 20 in the UnaG fluorescence assay. The top row (shaded in gray) of table 1310 is the TBIL value for each sample tested (i.e., 1.2 (4.0)=TBIL value for plasma sample 1; 5.5=TBIL value for plasma sample 2; etc.). The percent bias from control data (i.e., no surfactant) for the reactions is the average of the replicate values.

FIG. 15A is a plot showing the RFU over time for the n=2 reactions using the 13.1 mg/dL TBIL plasma sample and no surfactant control.

FIG. 15B is a plot showing the RFU over time for the n=2 reactions using the 13.1 mg/dL TBIL plasma and 0.1% Tween® 20.

FIG. 15C is a plot showing the RFU over time for the n=2 reactions using the 13.1 mg/dL TBIL plasma and 0.1% Tween® 80.

### 6.3.7. FINDER® Panel 1 Assays Using Tween® 80 and Facade®-TEM

In a multiplexed on-cartridge assay for newborn screening, a whole blood sample is typically split on-cartridge into two or more aliquots for performing different biochemical tests. In one example, the FINDER® Panel 1 cartridge includes tests for glucose-6-phosphate dehydrogenase (G6PD), albumin, and total bilirubin (TBIL). The FINDER® cartridge includes all sample preparation and assay reagents.

The FINDER® assays include multiple electrowetting operations such as dispensing, splitting, transporting, merging, and mixing to manipulate sample and reagent droplets. To determine whether Tween® 80, Facade-TEM interfere with FINDER® Panel 1 assays and/or generate electrowetting or fluidic failures (e.g., as evidenced by a change in droplet size) which could contribute to noise in an assay, the full assay protocol was run.

Briefly, uncovered FINDER® cartridges were loaded with either regular diluent, or 0.006% Tween® 80, or 0.05-mM Facade-TEM as diluent. A whole blood sample spiked with a plasma sample known to have a high TBIL level was loaded onto each cartridge. A cartridge was inserted into a FINDER® instrument and the full panel 1 protocol for sample preparation, G6PD, albumin, and TBIL was run.

FIG. 16 is a table 1600 showing the assay values for G6PD, albumin (ALB), and TBIL for the Tween® 20, Tween® 80, and Facade-TEM runs. All assay values were within expected range and noise. No run “flags” were triggered for any of the three runs indicating that all electrowetting operations were as expected.

### 6.3.8. GOD-POD-UnaG Assay Using Tween® 80 and Facade-TEM

To determine whether Tween® 80 or Facade-TEM interfere with the GOD-POD-UnaG assay (e.g., at the peroxidase reaction or the stop step), the full GOD-POD-UnaG assay protocol was performed on-bench.

Briefly, the on-bench GOD-POD-UnaG assay was performed using 4 plasma samples with known TBIL values ranging from 5.1 to 14 mg/dL. Plasma samples were diluted in glucose buffer with or without final surfactant (i.e., control samples) concentrations of 0.1% Tween® 20 (used as positive interference control), 0.006% Tween® 80, or 0.05-mM Facade-TEM. Enzyme reagents (i.e., glucose oxidase (GOD) and peroxidase (POD) and stop reagent (i.e., 1% ascorbic acid) were prepared in glucose buffer with or without final surfactant (i.e., control samples) concentrations of 0.1% Tween® 20 (used as positive interference control), 0.006% Tween® 80-, or 0.05-mM Facade-TEM. UnaG was diluted to 20 μM in a phosphate buffer with no surfactant. Samples and enzyme reagents were combined and incubated for a period of time. At the end of the incubation period, the reactions were stopped by the addition of a stop buffer and transferred to individual wells of a microtiter plate. UnaG was then aliquoted using a multichannel pipette into each sample well and fluorescence read after about 1 minute to about 20 minutes. In this example, fluorescence was read at t=3 minutes.

FIG. 17 is a table 1700 showing the percent bias from control for Tween® 20, Tween® 80, and Facade®-TEM in the GOD-POD-UnaG fluorescence assay. The left column of table 1700 is the assigned TBIL value for each sample tested (i.e., 5.1 mg/dL=TBIL value for plasma sample 1; 14 mg/dL=TBIL value for plasma sample 2; etc.). The data show that percent bias from no detergent control (calculated as control-reacted RFU values for all samples±detergent) for Tween® 80 is within 5% for all samples, demonstrating that Tween® 80 at 0.006% does not interfere with UnaG fluorescence, bilirubin oxidation by POD, or the ability to stop the oxidation reaction using 1% ascorbic acid. The data also shows that bias from control for Facade®-TEM was as high as 20%.

For comparison, values from the GOD-POD-UnaG fluorescence assay (“Control-Reacted RFU”; y-axis) were plotted against assigned values (x-axis) on the Arrow UB Analyzer (Arrows Co., Osaka, Japan). The “Control-Reacted RFU” is the RFU from a control reaction (which measures total unconjugated bilirubin) with the RFU from the GOD-POD-UnaG oxidation reaction subtracted. The “Control-Reacted RFU” values provide a surrogate measure for the unbound bilirubin present in the sample. The Arrow UB Analyzer is used to test for bilirubin that is not bound to albumin but does not specifically detect unconjugated bilirubin. The assigned unbound bilirubin (“Arrows UB (μg/dL)”) were 0.28, 0.95, 1.67, and >3.0 μg/dL, respectively.

FIG. 18A is a plot 1800 showing a comparison of the RFU (control-reacted sample) values (“Control”) relative to the Arrow UB Analyzer assigned value for Tween® 20. Tween 20 data points are indicated by an arrow.

FIG. 18B is a plot 1810 showing a comparison of the RFU (control-reacted sample) values (“Series 1”) relative to the Arrow UB Analyzer assigned value for Tween® 80 (“Series 2”). Tween 80 data points that do not overlap control data points are indicated by an arrow.

FIG. 18C is a plot 1820 showing a comparison of the RFU (control-reacted sample) values (“Control”) relative to the Arrow UB Analyzer assigned value for Facade®-TEM. Facade-TEM data points that do not overlap control data points are indicated by an arrow.

FIG. 19 is an enlargement of plot 1810 showing the comparison of the lower three data points for the RFU (control-reacted sample) values (plot line 1910) relative to the Arrow UB Analyzer assigned value for Tween® 80 (plot line 1920). Control: y=2637.1x+1149.2, R²=0.9928; Tween 80: y=2553.5x+1091.6, R²=0.992.

### 6.3.9. Methods Comparison Using Tween® 80 and Tween® 20

A comparison between the on-bench GOD-POD-UnaG assay and an on-cartridge GOD-POD-UnaG assay was performed using 7 plasma samples with known TBIL values ranging from 0.3 to 23.7 mg/dL. The on-bench assay was performed in the absence of surfactant. The on-cartridge assays were performed using Tween® 20 and Tween® 80. In an on-cartridge assay, the concentration of Tween® 20 may be from about 0.01% to about 1%, and the concentration of Tween® 80 may be from about 0.006% to about 0.024%. In this example, the concentrations used in the on-cartridge assay were 0.1% Tween® 20 and 0.006% Tween® 80.

FIG. 20 is a table 2000 showing the RFU values for the GOD-POD-UnaG assays performed on-cartridge and the “control-reacted” RFU values for the GOD-POD-UnaG assay performed on-bench. The TBIL plasma samples and their TBIL values are shown in the “Sample” column. The raw RFU values for the on-cartridge assay performed using Tween® 20 and Tween® 80 are shown in the second and third columns of table 2000 (i.e., “Tween 20 RFU” and “Tween 80 RFU”). A correction factor for instrument-dependent fluorescence variability was applied to the RFU data obtained for the Tween® 20 and Tween® 80 assays and are shown in the fourth and fifth columns of table 2000 (i.e., “Tween 20 C_RFU” and “Tween 80 C_RFU”). The control-reacted RFU values for the GOD-POD-UnaG assay performed on-bench (with no added surfactant) are shown in the column labeled “Bench”.

FIG. 21A is a plot 2100 showing the RFU (control-reacted sample) values vs DLS TBIL obtained on-cartridge using Tween® 20 and Tween® 80. The data was plotted using a correction for instrument-dependent fluorescence variability applied.

FIG. 21B is a plot 2110 showing the RFU (control-reacted sample) values obtained on-cartridge using Tween® 20 and Tween® 80 relative to the RFU obtained in the on-bench assay. The data was plotted using a correction for instrument-dependent fluorescence variability applied.

Although the foregoing subject matter has been described in some detail by way of illustration and example for purposes of clarity of understanding, it will be understood by those skilled in the art that certain changes and modifications can be practiced within the scope of the appended claims.

We claim:

1. A method for assaying analytes in a blood sample, the method comprising:
a. loading a blood sample comprising one or more analytes to be assayed onto a microfluidic device;
b. combining the blood sample with a buffer reagent comprising a surfactant to provide a diluted blood sample, wherein the surfactant is selected to permit electrowetting to conduct droplet operations using the blood sample;
c. dispensing one or more sample droplets from the diluted blood sample in a droplet operations gap of the microfluidic device, the gap comprising an oil filler fluid, thereby providing a diluted blood sample droplet;
d. transporting a diluted blood sample droplet to an assay reaction zone;
e. initiating a biochemical assay; and
f. optionally, repeating steps (d) and (e) one or more times.

2. The method of claim 1 wherein the blood sample comprises a whole blood sample.

3. The method of claim 1 further comprising processing the diluted blood sample to provide a processed blood sample comprising one or more analytes to be assayed.

4. The method of claim 3 wherein the processing comprises lysing the diluted blood sample.

5. The method of claim 3 wherein the processed blood sample comprises a blood component.

6. The method of claim 5 wherein the blood component comprises plasma.

7. The method of claim 1 wherein the buffer reagent further comprises a glucose reagent.

8. The method of claim 1 wherein the surfactant provides for electrowetting a whole blood sample droplet without causing significant lysis of the whole blood sample.

9. The method of claim 1 wherein the surfactant is selected to minimize or eliminate a fluorescence signal.

10. The method of claim 1 wherein the surfactant comprises a non-ionic surfactant.

11. The method of claim 10 wherein the non-ionic surfactant comprises Tween® 80.

12. The method of claim 10 wherein the non-ionic surfactant comprises Facade®-TEM.

13. The method of claim 1 wherein the surfactant comprises a zwitterionic surfactant.

14. The method of claim 13 wherein the zwitterionic surfactant comprises 11:0 Lyso PC.

15. The method of claim 1 wherein the biochemical assay is an enzymatic, fluorescence-based assay for measuring unbound bilirubin in a plasma droplet.

16. A method of measuring unbound bilirubin in a plasma droplet, the method comprising:
a. providing a plasma droplet comprising a glucose buffer and a surfactant compatible with performing a fluorescence-based unbound bilirubin assay using electrowetting-mediated droplet operations to perform assay steps;
b. splitting the plasma droplet into at least three sample droplets and initiating the unbound bilirubin assay, wherein:
i. a first sample droplet is combined with a buffer droplet to provide a control reaction droplet;
ii. a second sample droplet is combined with an enzyme reagent droplet to provide a short reaction droplet; and
iii. a third sample droplet is combined with a second enzyme reagent droplet to provide a long reaction droplet;
c. combining the control reaction droplet and the short reaction droplet with a stop reaction droplet at a time t1 to provide a control reacted droplet and a short-reacted droplet, wherein any unbound bilirubin in the short-reacted droplet is oxidized, thereby providing a t1 decomposition product droplet;
d. combining the long reaction droplet with a stop reaction droplet at a time t2 to provide a long-reacted droplet, wherein any remaining unbound bilirubin in the long-reacted droplet is oxidized, thereby providing a t2 decomposition product droplet;
e. diluting the control reacted droplet, t1 decomposition product droplet and t2 decomposition product droplet with a buffer reagent to provide a diluted control reacted droplet, a diluted t1 decomposition product droplet, and a diluted t2 decomposition product droplet for combining with a detection reagent;
f. combining the diluted control reacted, t1 decomposition product, and t2 decomposition product droplets with a detection reagent to provide a control/detection reagent droplet, a short reacted/detection reagent droplet and a long reacted/detection reagent droplet; and
g. detecting a reaction product in the control/detection reagent droplet, short reacted/detection reagent droplet, and long reacted/detection reagent droplet to determine the amount of unbound bilirubin in the plasma droplet.

17. The method of claim 16 wherein the enzyme reagent droplet comprises glucose oxidase (GOD) and peroxidase (POD).

18. The method of claim 16 wherein the stop reaction droplet comprises ascorbic acid.

19. The method of claim 16 wherein time t1 is about 48 seconds.

20. The method of claim 16 wherein the time t2 is about 120 seconds.

21. The method of claim 16 wherein combining each diluted reaction droplet with a detection reagent comprises transporting a diluted reaction droplet to a certain droplet operations electrode comprising a dried detection reagent and reconstituting the dried detection reagent.

22. The method of claim 16 wherein the dried detection reagent for detecting unbound bilirubin is UnaG.

23. The method of claim 16 wherein detecting a reaction product comprises measuring a UnaG fluorescence signal.

24. The method of claim 16 wherein determining the amount of unbound bilirubin in the plasma droplet comprises determining the difference in the UnaG fluorescence signal between the control/detection reagent droplet, and short and long reacted/detection reagent droplets.

25. A method for assaying analytes in a blood sample, the method comprising:
a. dispensing one or more sample droplets from a blood sample or diluted blood sample in a droplet operations gap of the microfluidic device;
b. initiating a biochemical assay on each of the oner or more sample droplets to detect unbound bilirubin in the diluted blood sample droplet.

26. The method of claim 25 wherein the sample droplets each have a volume less than about 5 mL.

27. The method of claim 25 wherein the microfluidic device comprises an electrowetting cartridge and the loading, combining, dispensing, and/or initiating is performed using electrowetting-mediated droplet operations.

28. The method of claim 25 wherein the blood sample is whole blood, plasma or serum.

29. A system comprising a computer processor and an electrowetting cartridge wherein the processor is programmed to execute the method of any one of the foregoing claims.

30. A kit comprising an electrowetting cartridge and reagents sufficient to execute the method of any one of the foregoing claims.
